SEQUENCE LISTING (37 sequences)

SEQ ID NO: 1 - protein, 167 aa, Artificial Sequence
  Sequence motif prepared based on the amino acid sequence of human ZG16 and the amino acid sequences of ZG16 for 19 different animal species showing a sequence identity of 80% or more with the human ZG16 amino acid sequence.
  misc_feature (2)..(2): Xaa is L or W
  misc_feature (3)..(3): Xaa is T, A, or I
  misc_feature (4)..(4): Xaa is V, I, A, L, or T
  misc_feature (5)..(5): Xaa is A, V, or T
  misc_feature (6)..(6): Xaa is L or I
  misc_feature (7)..(7): Xaa is L or I
  misc_feature (8)..(8): Xaa is A or V
  misc_feature (10)..(10): Xaa is L, F, or V
  misc_feature (12)..(12): Xaa is A or V
  misc_feature (13)..(13): Xaa is S or F
  misc_feature (14)..(14): Xaa is A, T, or V
  misc_feature (15)..(15): Xaa is S or E
  misc_feature (16)..(16): Xaa is G, S, A, or V
  misc_feature (17)..(17): Xaa is N, T, S, K, D, or deleted
  misc_feature (18)..(18): Xaa is A, S, E, or deleted
  misc_feature (19)..(19): Xaa is I, V, or S
  misc_feature (21)..(21): Xaa is A, S, P, or V
  misc_feature (22)..(22): Xaa is R, K, or Q
  misc_feature (23)..(23): Xaa is S, T, or A
  misc_feature (27)..(27): Xaa is S or N
  misc_feature (29)..(29): Xaa is E or D
  misc_feature (30)..(30): Xaa is Y or F
  misc_feature (33)..(33): Xaa is G, K, D, R, or S
  misc_feature (36)..(36): Xaa is K, E, G, or Q
  misc_feature (40)..(40): Xaa is H or Q
  misc_feature (43)..(43): Xaa is N or Y
  misc_feature (46)..(46): Xaa is D or E
  misc_feature (52)..(52): Xaa is L, I, or F
  misc_feature (54)..(54): Xaa is V or I
  misc_feature (56)..(56): Xaa is V or I
  misc_feature (57)..(57): Xaa is N or S
  misc_feature (58)..(58): Xaa is T, R, N, G, K, or S
  misc_feature (59)..(59): Xaa is Y or deleted
  misc_feature (62)..(62): Xaa is V or I
  misc_feature (70)..(70): Xaa is K or T
  misc_feature (71)..(71): Xaa is V or E
  misc_feature (74)..(74): Xaa is D, N, or A
  misc_feature (75)..(75): Xaa is Y, H, or F
  misc_feature (79)..(79): Xaa is R, N, T, S, or K
  misc_feature (80)..(80): Xaa is N, Q, S, G, or L
  misc_feature (82)..(82): Xaa is D or N
  misc_feature (84)..(84): Xaa is E or D
  misc_feature (89)..(89): Xaa is H or Y
  misc_feature (90)..(90): Xaa is P or S
  misc_feature (94)..(94): Xaa is V or I
  misc_feature (95)..(95): Xaa is I or V
  misc_feature (102)..(102): Xaa is K, E, or S
  misc_feature (103)..(103): Xaa is W, S, T, Y, K, G, R, N, or F
  misc_feature (105)..(105): Xaa is L or V
  misc_feature (106)..(106): Xaa is K or R
  misc_feature (107)..(107): Xaa is K or Q
  misc_feature (108)..(108): Xaa is L, M, or V
  misc_feature (109)..(109): Xaa is L, I, or V
  misc_feature (115)..(115): Xaa is G or F
  misc_feature (117)..(117): Xaa is Y or F
  misc_feature (119)..(119): Xaa is S, P, or A
  misc_feature (122)..(122): Xaa is K or T
  misc_feature (123)..(123): Xaa is D or A
  misc_feature (124)..(124): Xaa is S, T, I, or K
  misc_feature (129)..(129): Xaa is N or S
  misc_feature (131)..(131): Xaa is V, A, or L
  misc_feature (134)..(134): Xaa is H or Y
  misc_feature (142)..(142): Xaa is I or F
  misc_feature (146)..(146): Xaa is S or A
  misc_feature (147)..(147): Xaa is G or S
  misc_feature (148)..(148): Xaa is S, A, or I
  misc_feature (149)..(149): Xaa is L, A, F, or V
  misc_feature (151)..(151): Xaa is D or N
  misc_feature (152)..(152): Xaa is A or S
  misc_feature (154)..(154): Xaa is G or S
  misc_feature (155)..(155): Xaa is L or F
  misc_feature (159)..(159): Xaa is V, T, L, or S
  misc_feature (162)..(162): Xaa is S or deleted
  misc_feature (163)..(163): Xaa is S, H, D, E, N, I, T, V, or deleted
  misc_feature (164)..(164): Xaa is C, Y, or deleted
  misc_feature (165)..(165): Xaa is S, N, E, G, or deleted
  misc_feature (166)..(166): Xaa is R, T, S, K, or deleted
  misc_feature (167)..(167): Xaa is C or deleted
MXXXXXXXLXCXXXXXXXXQXXXSSYXGXXGGXGGXRFSXSGXQLXGPITAXRXRXXXXYIXGLQVRYGXXWSXXVGGXXGXLXEIFLXXGESXXQVSGKYXXYXXXXXFVTDKXRXLXFGXXXGTSFXAXPLXPNTVLRFXSGRXXXXIXXIXXHWDXYPXXXXXX

SEQ ID NO: 2 - protein, 167 aa, Artificial Sequence
  Sequence motif prepared based on the amino acid sequence of human ZG16 and the amino acid sequences of ZG16 for 15 different animal species showing a sequence identity of 84% or more with the human ZG16 amino acid sequence.
  misc_feature (3)..(3): Xaa is T, A, or I
  misc_feature (4)..(4): Xaa is V or I
  misc_feature (5)..(5): Xaa is A or T
  misc_feature (6)..(6): Xaa is L or I
  misc_feature (7)..(7): Xaa is L or I
  misc_feature (8)..(8): Xaa is A or V
  misc_feature (10)..(10): Xaa is L or V
  misc_feature (14)..(14): Xaa is A or V
  misc_feature (16)..(16): Xaa is G, A, or V
  misc_feature (17)..(17): Xaa is N, S, K, or D
  misc_feature (18)..(18): Xaa is A or S
  misc_feature (19)..(19): Xaa is I or V
  misc_feature (21)..(21): Xaa is A, S, or V
  misc_feature (22)..(22): Xaa is R or Q
  misc_feature (23)..(23): Xaa is S, T, or A
  misc_feature (27)..(27): Xaa is S or N
  misc_feature (29)..(29): Xaa is E or D
  misc_feature (30)..(30): Xaa is Y or F
  misc_feature (33)..(33): Xaa is G, K, D, R, or S
  misc_feature (36)..(36): Xaa is K, E, G, or Q
  misc_feature (43)..(43): Xaa is N or Y
  misc_feature (46)..(46): Xaa is D or E
  misc_feature (52)..(52): Xaa is L, I, or F
  misc_feature (54)..(54): Xaa is V or I
  misc_feature (56)..(56): Xaa is V or I
  misc_feature (57)..(57): Xaa is N or S
  misc_feature (58)..(58): Xaa is T, R, N, G, or K
  misc_feature (59)..(59): Xaa is Y or deleted
  misc_feature (62)..(62): Xaa is V or I
  misc_feature (70)..(70): Xaa is K or T
  misc_feature (74)..(74): Xaa is D, N, or A
  misc_feature (75)..(75): Xaa is Y or H
  misc_feature (79)..(79): Xaa is R, N, T, S, or K
  misc_feature (80)..(80): Xaa is N, Q, S, G, or L
  misc_feature (82)..(82): Xaa is D or N
  misc_feature (102)..(102): Xaa is K or E
  misc_feature (103)..(103): Xaa is W, S, Y, K, G, R, N, or F
  misc_feature (105)..(105): Xaa is L or V
  misc_feature (106)..(106): Xaa is K or R
  misc_feature (107)..(107): Xaa is K or Q
  misc_feature (108)..(108): Xaa is L, M, or V
  misc_feature (109)..(109): Xaa is L, I, or V
  misc_feature (119)..(119): Xaa is S, P, or A
  misc_feature (122)..(122): Xaa is K or T
  misc_feature (123)..(123): Xaa is D or A
  misc_feature (124)..(124): Xaa is S, T, or I
  misc_feature (129)..(129): Xaa is N or S
  misc_feature (131)..(131): Xaa is V or A
  misc_feature (134)..(134): Xaa is H or Y
  misc_feature (146)..(146): Xaa is S or A
  misc_feature (147)..(147): Xaa is G or S
  misc_feature (148)..(148): Xaa is S, A, or I
  misc_feature (149)..(149): Xaa is L, A, F, or V
  misc_feature (151)..(151): Xaa is D or N
  misc_feature (152)..(152): Xaa is A or S
  misc_feature (154)..(154): Xaa is G or S
  misc_feature (159)..(159): Xaa is V, T, or S
  misc_feature (162)..(162): Xaa is S or deleted
  misc_feature (163)..(163): Xaa is S, H, D, N, I, T, V, or deleted
  misc_feature (164)..(164): Xaa is C or deleted
  misc_feature (165)..(165): Xaa is S, N, G, or deleted
  misc_feature (166)..(166): Xaa is R, T, S, K, or deleted
  misc_feature (167)..(167): Xaa is C or deleted
MLXXXXXXLXCASXSXXXXQXXXSSYXGXXGGXGGXRFSHSGXQLXGPITAXRXRXXXXYIXGLQVRYGXVWSXXVGGXXGXLEEIFLHPGESVIQVSGKYXXYXXXXXFVTDKGRYLXFGXXXGTSFXAXPLXPNTVLRFISGRXXXXIXXIXLHWDXYPXXXXXX

SEQ ID NO: 3 - protein, 167 aa, Artificial Sequence
  Sequence motif prepared based on the amino acid sequence of humanZG16 and the amino acid sequences of ZG16 for 6 different animal species showing a sequence identity of 90% or more with the human ZG16 amino acid sequence.
  misc_feature (3)..(3): Xaa is T or I
  misc_feature (4)..(4): Xaa is V or I
  misc_feature (5)..(5): Xaa is A or T
  misc_feature (6)..(6): Xaa is L or I
  misc_feature (14)..(14): Xaa is A or V
  misc_feature (16)..(16): Xaa is G or A
  misc_feature (17)..(17): Xaa is N or D
  misc_feature (19)..(19): Xaa is I or V
  misc_feature (27)..(27): Xaa is S or N
  misc_feature (36)..(36): Xaa is K or Q
  misc_feature (57)..(57): Xaa is N or S
  misc_feature (58)..(58): Xaa is T, R, N, or K
  misc_feature (70)..(70): Xaa is K or T
  misc_feature (79)..(79): Xaa is R, S, or K
  misc_feature (80)..(80): Xaa is N or S
  misc_feature (103)..(103): Xaa is W, G, S, or N
  misc_feature (106)..(106): Xaa is K or R
  misc_feature (108)..(108): Xaa is L or V
  misc_feature (109)..(109): Xaa is L or V
  misc_feature (119)..(119): Xaa is S, P, or A
  misc_feature (124)..(124): Xaa is S or T
  misc_feature (146)..(146): Xaa is S or A
  misc_feature (149)..(149): Xaa is L or V
  misc_feature (162)..(162): Xaa is S or deleted
  misc_feature (163)..(163): Xaa is S, I, or deleted
  misc_feature (164)..(164): Xaa is C or deleted
  misc_feature (165)..(165): Xaa is S or deleted
  misc_feature (166)..(166): Xaa is R, S, K, or deleted
  misc_feature (167)..(167): Xaa is C or deleted
MLXXXXLALLCASXSXXAXQARSSSYXGEYGGGGGXRFSHSGNQLDGPITALRVRVXXYYIVGLQVRYGXVWSDYVGGXXGDLEEIFLHPGESVIQVSGKYKXYLXKXXFVTDKGRYLXFGKDXGTSFNAVPLHPNTVLRFISGRXGSXIDAIGLHWDVYPXXXXXX

SEQ ID NO: 4 - protein, 167 aa, Artificial Sequence
  Consensus sequence.
MLTVALLALLCASASANAIQARSSSYSGEYGGGGGKRFSHSGNQLDGPITALRVRVNRYYIVGLQVRYGKVWSDYVGGTSGDLEEIFLHPGESVIQVSGKYKSYLRKLVFVTDKGRYLPFGKDTGTSFNAVPLHPNTVLRFISGRSGSLIDAIGLHWDVYPSDCSSC

SEQ ID NO: 5 - protein, 167 aa, Homo sapiens
  misc_feature (32)..(32): Xaa is G or S
  misc_feature (109)..(109): Xaa is V or L
  misc_feature (162)..(162): Xaa is S or T
MLTVALLALLCASASGNAIQARSSSYSGEYGXGGGKRFSHSGNQLDGPITALRVRVNTYYIVGLQVRYGKVWSDYVGGRNGDLEEIFLHPGESVIQVSGKYKWYLKKLXFVTDKGRYLSFGKDSGTSFNAVPLHPNTVLRFISGRSGSLIDAIGLHWDVYPXSCSRC

SEQ ID NO: 6 - protein, 167 aa, Homo sapiens
  misc_feature: UniProt entry 060844
MLTVALLALLCASASGNAIQARSSSYSGEYGGGGGKRFSHSGNQLDGPITALRVRVNTYYIVGLQVRYGKVWSDYVGGRNGDLEEIFLHPGESVIQVSGKYKWYLKKLLFVTDKGRYLSFGKDSGTSFNAVPLHPNTVLRFISGRSGSLIDAIGLHWDVYPSSCSRC

SEQ ID NO: 7 - protein, 167 aa, Homo sapiens
  misc_feature: NCBI Reference Sequence NP_689551.1
MLTVALLALLCASASGNAIQARSSSYSGEYGSGGGKRFSHSGNQLDGPITALRVRVNTYYIVGLQVRYGKVWSDYVGGRNGDLEEIFLHPGESVIQVSGKYKWYLKKLVFVTDKGRYLSFGKDSGTSFNAVPLHPNTVLRFISGRSGSLIDAIGLHWDVYPTSCSRC

SEQ ID NO: 8 - protein, 167 aa, Homo sapiens
  misc_feature: NCBI Reference Sequence NP_689551.2
MLTVALLALLCASASGNAIQARSSSYSGEYGGGGGKRFSHSGNQLDGPITALRVRVNTYYIVGLQVRYGKVWSDYVGGRNGDLEEIFLHPGESVIQVSGKYKWYLKKLVFVTDKGRYLSFGKDSGTSFNAVPLHPNTVLRFISGRSGSLIDAIGLHWDVYPTSCSRC

SEQ ID NO: 9 - protein, 167 aa, Rattus norvegicus
MLAIALLVLLCASASANSIQSRSSSYSGEYGGKGGKRFSHSGNQLDGPITAIRIRVNRYYIIGLQVRYGTVWSDYVGGNQGDLEEIFLHPGESVIQVSGKYKSYVKQLIFVTDKGRYLPFGKDSGTSFNAVPLHPNTVLRFISGRSGSAIDAISLHWDTYPSHCNTC

SEQ ID NO: 10 - protein, 167 aa, Mus musculus
MLAVALLVLLCASASANSIQSRTSSYSGEYGGKGGKRFSHSGNQLDGPITAFRIRVNRYYIVGLQVRYGTVWSDYVGGTQGDLEEIFLHPGESVIQVSGKYKSYVKQMIFVTDKGRYLPFGKASGTSFNAVPLHPNTVLRFISGRSGSAIDSISLHWDTYPSHCNTC

SEQ ID NO: 11 - protein, 167 aa, Bos taurus
MLTAALLALLCASASATEIQPRASSYSGEYGGGGGERFSQSGNQLDGPITAIRIRVSNYYIVGLQVRYGTVWSDYVGGTSGDLDEIFLYPGESIVQVSGKYKTYLRKLVFVTDKFRFLSFGKDTGTSFNAVPLYPNTVLRFISGRAGSLIDAIGFHWDLYPSDYESC

SEQ ID NO: 12 - protein, 167 aa, Pan troglodytes
MLTVALLALLCASASGNAIQARSSSYSGEYGGGGGKRFSHSGNQLDGPITALRVRVNTYYIVGLQVRYGKVWSDYVGGRNGDLEEIFLHPGESVIQVSGKYKWYLKKLVFVTDKGRYLSFGKDSGTSFNAVPLHPNTVLRFISGRSGSLIDAIGLHWDVYPSSCSRC

SEQ ID NO: 13 - protein, 165 aa, Sarcophilus harrisii
MLALVLLALFCVSAESVQPRSSSYNGEYGGGGGERFSHSGNQLDGPITAIRIRVNRYYIVGLQVRYGKEWSNYVGGSQGDLEEIFLYPGESIIQVSGKYKYYVRKLVFVTDKGRYLPFGKDTGTSFNAAPLYPNTVLRFFSGRSGSLINAIGLHWDVYPSECSSC

SEQ ID NO: 14 - protein, 167 aa, Felis catus
MLTIALLALVCASASANAIQARSSSYNGEYGGDGGERFSHSGYQLEGPITAIRIRINRYYIVGLQVRYGKVWSDYVGGTQGDLEEIFLHPGESVIQVSGKYKYYLRKLVFVTDKGRYLPFGKDTGTSFNAVPLYPNTVLRFISGRSGALINAIGLHWDVYPSDCGSC

SEQ ID NO: 15 - protein, 166 aa, Equus caballus
MLTIALLALLCASASASAIQARSSSYNGDFGGRGGGRFSHSGNQLDGPITALRIRVNGYIIGLQVRYGKVWSAHVGGTGGNLEEIFLHPGESVIQVSGKYEKYLRKLVFVTDKGRYLSFGTDIGTSFSAVPLHPNTVLRFISGRAGSLIDAIGLHWDVYPSNCSSC

SEQ ID NO: 16 - protein, 167 aa, Cavia porcellus
MLITVLLVLLCAFTSAKASQVKASSYNGEYGGSGGKRFSHSGYQLEGPITALRIRVNKYYIVGLQVRYGKVWSDFVGGKSGDLEEIFLHSGESVIQVSGKYSSYLRKLVFVTDKGRYLSFGKDTGTSFNALPLYPNTVLRFISGRSGSFIDAIGLHWDSYPSDCSSC

SEQ ID NO: 17 - protein, 167 aa, Macaca mulatta
MLTVALLALLCASASANAIQARSSSYSGEYGGGGGKRFSHSGNQLDGPITALRVRVNNYYIVGLQVRYGKVWSDYVGGRSGDLEEIFLHPGESVIQVSGKYKGYLKKVVFVTDKGRYLAFGKDSGTSFNAVPLHPNTVLRFISGRSGSVIDAIGLHWDVYPSICSSC

SEQ ID NO: 18 - protein, 167 aa, Canis familiaris
MLTIALLALLCASASANAIQARSSSYNGEYGGGGGQRFSHSGNQLEGPITALRVRVNRYYIVGLQVRYGKVWSDYVGGTQGDLEEIFLHPGESVIQVSGKYKYYLRKLVFVTDKGRYLPFGKDTGTSFNAVPLYPNTVLRFISGRASSLINAIGLHWDVYPSDCSSC

SEQ ID NO: 19 - protein, 167 aa, Callithrix jacchus
MLTVALLALLCASASASAIQARSSSYSGEYGGGGGERFSHSGNQLDGPITAFRVRVSKYYIIGLQVRYGKVWSNYVGGSSGDLEEIFLHPGESVIQVSGKYKRYLRKLVFVTDKGRYLPFGKDTGTSFNAVPLHPNTVLRFISGRSGAVIDAIGLHWDVYPSTCSSC

SEQ ID NO: 20 - protein, 167 aa, Pongo abelii
MLTVALLALLCASVSANAIQARSSSYSGEYGGGGGKRFSHSGNQLDGPITALRVRVNKYYIVGLQVRYGKVWSDYVGGSNGDLEEIFLHPGESVIQVSGKYKWYLKKLVFVTDKGRYLPFGKDSGTSFNAVPLHPNTVLRFISGRSGSLIDAIGLHWDVYPSSCSRC

SEQ ID NO: 21 - protein, 167 aa, Gorilla gorilla gorilla
MLTVALLALLCASASGNAIQARSSSYSGEYGGGGGKRFSHSGNQLDGPITALRVRVNTYYIVGLQVRYGKVWSDYVGGSNGDLEEIFLHPGESVIQVSGKYKWYLKKLVFVTDKGRYLSFGKDSGTSFNAVPLHPNTVLRFISGRSGSLIDAIGLHWDVYPSSCSRC

SEQ ID NO: 22 - protein, 167 aa, Otolemur garnetti
MLTIALIALLCASASANAIQARSSSYSGEYGGGGGKRFSHSGNQLEGPITAIRVRVNRYYIVGLQVRYGKVWSDYVGGTSGDLEEIFLHPGESVIQVSGKYKNYLRKMVFVTDKGRYLPFGKDTGTSFNAVPLHPNTVLRFISGRAGSVIDAIGLHWDVYPSDCSSC

SEQ ID NO: 23 - protein, 167 aa, Mustela putorius furo
MLAIALLALLCASASAKAIQARASSYNGEYGGGGGERFSHSGYQLEGPITAIRVRVNRYYIVGLQVRYGKVWSDYVGGTQGDLEEIFLHPGESVIQVSGKYKYYLRKLVFVTDKGRYLPFGKDTGTSFNAAPLYPNTVLRFISGRSGSLINAIGLHWDSYPSDCGSC

SEQ ID NO: 24 - protein, 167 aa, Ovis aries
MWTTALLALLCASASATEIQPRASSYSGEYGGGGGERFSQSGNQLDGPITAIRIRVSSYYIVGLQVRYGTVWSDYVGGTSGDLDEIFLYPGESIVQVSGKYKTYLRKLVFVTDKFRFLSFGTDKGTSFNAVPLYPNTVLRFISGRSGSLIDAIGFHWDLYPSDYESC

SEQ ID NO: 25 - protein, 167 aa, Loxodonta africana
MLTIALLALLCASASVNAIQVQSSSYNGEYGGSGGQRFSHSGNQLEGPITAIRVRVNRYYIVGLQVRYGKVWSDYVGGTLGDLEEIFLHPGESVIQVSGKYKFYLRKLVFVTDKGRYLPFGKDTGTSFNAAPLYPNTVLRFISGRSSIFINAIGLHWDVYPSVCSSC

SEQ ID NO: 26 - protein, 167 aa, Nomascus leucogenys
MLIVTLLALLCASASADAVQARSSSYSGEYGGGGGKRFSHSGNQLDGPITALRVRVNKYYIVGLQVRYGTVWSDYVGGRNGDLEEIFLHPGESVIQVSGKYKSYLKKLVFVTDKGRYLPFGKDSGTSFNAVPLHPNTVLRFISGRSGSVIDAIGLHWDVYPSSCSKC

SEQ ID NO: 27 - protein, 161 aa, Spermophilus tridecemlineatus
MLTIAILALLCASASANAIQARSSSYNGEYGGGGGQRFSHSGNQLDGPITALRVRVSRYYIVGLQVRYGKVWSDYVGGKSGDLEEIFLHPGESVIQVSGKYKNYLRKLVFVTDKGRYLPFGKDTGTSFNAVPLHPNTVLRFISGRAGSVIDAIGLHWDVYP

SEQ ID NO: 28 - DNA, 20 bp, Artificial Sequence
  Universal forward primer 27F
agagtttgatcmtggctcag

SEQ ID NO: 29 - DNA, 20 bp, Artificial Sequence
  Universal reverse primer 1492R
cggttaccttgttacgactt

SEQ ID NO: 30 - DNA, 20 bp, Artificial Sequence
  Universal 926F
aaactcaaakgaattgacgg

SEQ ID NO: 31 - DNA, 18 bp, Artificial Sequence
  Universal 1062R
ctcacrrcacgagctgac

SEQ ID NO: 32 - DNA, 20 bp, Artificial Sequence
  Bacteroidetes 798cfbF
craacaggattagataccct

SEQ ID NO: 33 - DNA, 19 bp, Artificial Sequence
  Bacteroidetes cfb967R
ggtaaggttcctcgcgtat

SEQ ID NO: 34 - DNA, 21 bp, Artificial Sequence
  Firmicutes 928F-Firm
tgaaactyaaaggaattgacg

SEQ ID NO: 35 - DNA, 17 bp, Artificial Sequence
  Firmicutes 1040FirmR
accatgcaccacctgtc

SEQ ID NO: 36 - DNA, 19 bp, Artificial Sequence
  Gamma-proteobacteria 1080gF
tcgtcagctcgtgtygtga

SEQ ID NO: 37 - DNA, 16 bp, Artificial Sequence
  Gamma-proteobacteria g1202R
cgtaagggccatgatg

(19) United States
(12) Patent Application Publication
(10) Pub. No.: US 2018/0028603 A1
(43) Pub. Date: Feb. 1, 2018

(54) A PROTEIN BASED ON ZG16 FOR USE AS A MEDICAMENT

(71) Applicants: Gunnar C. HANSSON, Göteborg (SE); Joakim BERGSTRÖM, Hovås (SE); George BIRCHENOUGH, Västra Frölunda (SE); Malin E.V. JOHANSSON, Västra Frölunda (SE)

(72) Inventors: Gunnar C. HANSSON, Göteborg (SE); Joakim BERGSTRÖM, Hovås (SE); George BIRCHENOUGH, Västra Frölunda (SE); Malin E.V. JOHANSSON, Västra Frölunda (SE)

(21) Appl. No.: 15/527,579

(22) PCT Filed: Nov. 20, 2015
(86) PCT No.: PCT/EP2015/077206

§ 371 Date: May 17, 2017

(30) Foreign Application Priority Data
Nov. 21, 2014 (EP) 14194364.7

(51) Int. Cl. A61K 38/17

(52) U.S. Cl. CPC A61K 38/1709

(57) ABSTRACT

A protein based on zymogen granule 16 protein (ZG16) for use in the prevention and/or treatment of a disorder related to a bacterial imbalance in the gastrointestinal tract is disclosed. The protein based on ZG16 may comprise the sequence motifs defined by any one of the sequences having SEQ ID NO:s 1-3, optionally excluding the amino acids in positions 1-20, or 1-16.

### TECHNICAL FIELD OF THE INVENTION

The present invention relates to the field of medicaments, in particular to a protein based on zymogen granule 16 protein (ZG16) for use in the prevention and treatment of a disorder related to a dysfunction in the gastrointestinal tract.

### BACKGROUND

Today there are many people suffering from different types of disorders related to dysfunctions in the gastrointestinal tract. Common examples are IBS (Irritable Bowel Syndrome), IBD (Inflammatory Bowel Disease), and various kinds of colitis, such as ulcerative colitis. Some of the disorders are chronic conditions which may have a large negative impact on the life of the person having the disorder. Metabolic disorders are also influenced by the microbiota.

The underlying causes of these disorders have not yet been fully understood, but it is generally assumed that the disorders are caused by a bacterial infection in the gastrointestinal tract and/or an immune system malfunction, which may trigger an excessive inflammation in the gastrointestinal tract.

Different methods of treatment have been suggested to treat and/or to reduce the symptoms of such disorders. Since some of the disorders are chronic conditions, the method of treatment may be a long term treatment. In order to treat an infection caused by bacteria, antibiotics may be administered. If the disorder is caused by an inflammation, anti-inflammatory drugs may be administered. Another approach to reduce inflammation may be to administer drugs comprising immune system suppressors which target the immune system. In severe cases, surgery might be performed, such as to remove parts of the intestine of the gastrointestinal tract.

Available treatment methods may not fully cure the disorders, and furthermore, available methods have drawbacks, such as side effects which may negatively influence the body.

Hence, there is still a need in the art for improved methods and compositions of preventing and/or treating disorders related to dysfunctions in the gastrointestinal tract.

### SUMMARY OF THE INVENTION

An object of the present invention is to overcome the above-mentioned drawbacks by providing uses and methods which allow for efficient prevention and/or treatment of disorders related to dysfunctions in the gastrointestinal tract.

These objects are achieved, in a first aspect, by means of a protein based on zymogen granule 16 protein (ZG16) for use in the prevention and/or treatment of a disorder which is related to a bacterial imbalance in the gastrointestinal tract.

It has surprisingly found that ZG16 can be used as a medicament due to its ability to bind to peptidoglycan which is present on the surface of Gram positive bacteria. ZG16 can thereby cause Gram positive bacteria to form aggregates, which in turn reduces penetration of the bacteria through intestinal mucus. As a result, the bacteria can be kept at a distance from the epithelium of the gastrointestinal tract, thereby avoiding diseases associated with an undesired contact between Gram positive bacteria and epithelium.

Thus, otherwise stated, the present invention relates to the use of a protein based on ZG16 in the prevention and/or treatment of a disorder caused by Gram positive bacteria coming into contact with the gastrointestinal epithelium.

An advantage of using a protein based on ZG16 is that ZG16 is a naturally occurring protein in for example the gastrointestinal tract. Since the protein is naturally occurring in the gastrointestinal tract, less negative side effects may be expected as compared to conventional treatments. Furthermore, experiments have shown that ZG16 is fairly resistant to trypsin degradation, thus it is expected that ZG16 may be orally administered. This is a significant advantage of the present invention considering the fact that proteins may rarely be administered by this route.

Examples of disorders which are related to a bacterial imbalance in the gastrointestinal tract, and which may be treated by the use of a protein based on ZG16 in accordance with the present invention are:
Gastrointestinal diseases, such as Irritable Bowel Syndrome (IBS), or gastrointestinal inflammatory diseases. Examples of gastrointestinal inflammatory diseases are systemic inflammatory diseases, or Inflammatory Bowel Disease (IBD), such as Crohn's disease or colitis, such as ulcerative colitis. Also contemplated are gastrointestinal diseases being intestinal symptoms of cystic fibrosis, e.g. DIOS.
Bacterial infections.
Disorders related to the metabolic syndrome, e.g. obesity and/or diabetes, such as type 2 diabetes.

The protein based on ZG16 may be comprised in a pharmaceutical composition comprising a pharmaceutically effective amount of protein based on ZG16. The pharmaceutical composition may be formulated for oral, nasal, rectal, topical, intravenous, intraartery, intracavitary, intramuscular, subcutaneous, or transdermal administration, and may be in the form of a tablet, a capsule, a suppository, a powder, a paste, a solution, a cream, a gel, and/or an administration unit comprising bacteria having the ability to produce and secrete a protein based on ZG16.

In a second aspect, the present invention relates to a method for the prevention and/or treatment of a disorder which is related to a bacterial imbalance in the gastrointestinal tract, comprising administering to a patient in need thereof a pharmaceutically effective amount of a protein based on ZG16. In particular, this aspect of the present invention encompasses methods for treatment of any one of the specific disorders mentioned above, by administering to a patient in need thereof a pharmaceutically effective amount of a protein based on ZG16.

A protein based on ZG16 for use according to the invention, or in a method according to the present invention, may comprise a sequence motif defined by any one of the sequences having SEQ ID NO:s 1-3, optionally excluding the amino acids in positions 1-20, or 1-16.

Alternatively, a protein based on ZG16 for use according to the invention, or in a method according to the present invention, may have a sequence identity of at least 80%, such as at least 84%, at least 90%, or at least 99% with human ZG16 as defined by SEQ ID NO. 6, optionally excluding the amino acids in positions 1-20, or 1-16.

Preferably, a protein based on ZG16 for use according to the invention, or in a method according to the present invention, is defined by SEQ ID NO:s 5-8, optionally excluding the amino acids in positions 1-20, or 1-16.

### DESCRIPTION OF THE DRAWINGS

FIG. 1. ZG16 binds peptidoglycan and Gram positive bacteria. Binding experiments were performed using a DELFIA based assay. (A) Recombinant ZG16-IgG at various concentrations was allowed to bind insoluble peptidoglycan in a 96-well plate. ZG16-IgG bound to the peptidoglycan after extensive washing compared to the control MUC1-IgG. Error bars represent standard error of the mean. (B) Preincubation with 30 mM MurNAc before partially inhibited binding of ZG16-IgG to peptidoglycan. (C) ZG16-IgG binding to the G+ bacteria Lactobacillus jensenii and not to Escherichia coli. The assay was performed in a similar way as for the peptidoglycan binding.

FIG. 2. ZG16 is not bactericidal, but aggregates bacteria and limits motility. (A) The growth of Lactobacillus jensenii was monitored for 24 h by OD⁵⁶⁰measurements in the presence or absence of recombinant rZG16. (B) Bacillus subtilis was incubated with up to 2 mM DTT in the presence of rZG16 in a radial diffusion assay; white dashed line represents application zone. (C) Syto9 stained Enterococcus faecalis and Escherichia coli cultures were incubated with BSA or rZG16 and bacteria were spread on microscopy slides and imaged by confocal microscopy; images show representative field views of BSA (upper panels) or rZG16 (lower panels) treated bacteria; scale bars 50 μm. (D) Fluorescent cell areas from the microscopy pictures calculated using Imaris software; statistical significance calculated using Tukey's multiple comparison test (ns, not significant; *p<0.001); data representative of n=3 independent experiments. (E) Growth of B. subtilis inoculated into the centre of low density agar treated with BSA or rZG16; images show representative bacterial growth on the agar (left panel) and 20 mm confocal micrographs of Syto9 stained bacteria within the agar (right panels); dashed white line represents the approximate BSA/rZG16 application area; grey square shows area where confocal micrographs were acquired; scale bar 1 cm. (F) Magnified images of Syto9 stained B. subtilis within low density agar from regions 1, 2 and 3 indicated on confocal micrographs shown in (E); scale bars 100 μm. (G) Normalised fluorescence of B. subtilis within BSA/rZG16 treated low density agar at different distances from the initial point of bacterial inoculation; dashed lines represent SEM from n=3 independent experiments.

FIG. 3. Mucus phenotype in Zg16^{-/-} mice. (A) Mucus measurement of WT (n=9) and ZG16^{-/-} mice (n=6). (B) Distal colon tissue was mounted in a horizontal Ussing chamber, charcoal was added to visualize the mucus layer and the distance to the epithelial cells measured. The secretagogue carbachol was added to one group after 30 min. Penetrability measurements of WT and ZG16^{-/-} mice.

FIG. 4. ZG16 alters distal colonic mucus penetration by Gram-positive bacteria in vitro. (A-E) C57BL/6 WT or Zg16^{-/-} distal colon tissues were mounted in an imaging chamber; Fluorescent beads were applied apically to visualise the interface between the impenetrable (IM) and penetrable (PM) mucus layers; 10⁸ CFU E. faecalis (Ef) or E. coli (Ec) were stained with BacLight Red then treated with 10 μg rZG16 and applied apically to mucus; 3D z-stacks of the mucus surface were acquired by confocal microscopy. (A) Schematic representation of data acquisition region covering the IM/PM interface. (B) Confocal z-stacks of WT mucus surface exposed to untreated Ef/Ec (left panels) or Ef/Ec treated with rZG16 (right panels); White dashed line indicates IM/PM interface. (C) Distribution of beads and untreated or rZG16 treated Ef (left graph) or Ec (right graph) along the z-stack z-axis at the WT IM/PM interface; Coloured dashed lines represent SEM from n=6 mice; Black dashed lines separate IM, interface and PM regions of the z-stack. (D) Quantification of control and rZG16 treated Ef and Ec from the PM z-stack region indicated by the arrows in (C); Error bars represent SEM from n=6 mice. (E) Confocal z-stacks of Zg16^{-/-} mucus surface exposed to untreated Ef/Ec (left panels) or Ef/Ec treated with rZG16 (right panels); White dashed line indicates IM/PM interface. All images are representative of n=6 mice; Scale bars are 20 μm; Statistical significance calculated with Tukey's multiple comparison test (ns, not significant; *p<0.05).

FIG. 5. ZG16 alters the mucus associated intestinal microbiota as well as the distribution and motile capacity of bacteria within the mucus. (A) Zg16^{+/+} and Zg16^{-/-} littermate bacterial 16S copy number detected in stool and unflushed (total mucus) or flushed (inner mucus) colonic tissue samples by qPCR. Data were normalized to stool mass or mucus volume. (B) Group-specific qPCR showing an increased abundance of Gram-positive Firmicutes in the total and inner mucus of the Zg16^{-/-} in relation to littermate Zg16^{+/+} mice. (C-E) Conventionally raised C57BL/6 (WT), germ-free (GF) and Zg16^{-/-} distal colon tissues were mounted in an imaging chamber; fluorescent beads were applied apically to visualize the interface between the impenetrable (IM) and penetrable (PM) mucus layers; bacteria and tissues were visualized in situ using the nucleic acid binding dye Syto9. Mucus, bacteria and tissues were imaged by confocal microscopy. (C) Schematic representation of chamber mounted colonic tissues with IM and PM layers, beads and bacteria indicated; Black dashed line represents the focal plane where confocal optical sections were acquired. (D) Confocal micrographs through WT, GF and Zg16^{-/-} colonic mucus; Upper dashed lines indicate the IM/PM interface; Lower dashed lines indicate the edge of the colonic tissue. (E) Magnified confocal micrographs from the inset boxes in D; Upper panel shows morphologically distinctive bacteria in the mucus; Lower panel shows bacteria (indicated by white arrows) near the tissue surface. (F-G) Bacterial motility in WT, Zg16^{-/-} and Zg16^{-/-}+rZG16 mucus was assessed by recording 1 min time series of optical sections through the colonic mucus as pictured in E. (F) Quantification of motile capacity (maximum detected velocity) of beads at the IM/PM interface, all bacteria within the mucus and only bacteria classed as motile within the mucus. (G) Proportion of bacteria determined as motile per imaging field. All scale bars are 50 μm; All error bars are SEM from n=9 Zg16^{+/+} and n=10 Zg16^{-/-} animals; Statistical significances calculated with Mann-Whitney U test (A), Sidak's (B, F) or Dunn's (G) multiple comparison tests (ns, not significant; *p<0.05).

FIG. 6. Zg16^{-/-} mice have an increased host systemic bacterial load, serum inflammatory marker levels, and increased body mass. (A-C) Mesenteric lymph node (MLN) and spleen tissue were acquired from C57BL/6 WT and Zg16^{-/-} mice; Tissues were weighed, homogenized and DNA was extracted. (A) Quantification of bacterial 16S copy number in MLN and spleen tissue by qPCR; Error bars are SEM, n=10 mice per group. (B) Estimation of the abundance of different bacterial taxonomic groups (Bacteroidetes, Firmicutes and Gamma-proteobacteria) in the tissues examined in (A) using qPCR. (C) Bacterial BHIS agar culture of spleen tissue homogenates from WT (left panel) and Zg16^{-/-} (right panel) mice. (D) Magnification of (C) showing multiple bacterial colony morphologies. (E-G) Quantification of serum TNFα, KC/GRO and IL-6 by MSD; Error bars are SEM from n=10 mice. (H) Total body mass of 14-week old male and female WT and Zg16^{-/-} mice; Error bars are SEM from n=4 mice per group. (I) Animals were weighed to determine total mass, after which abdominal fat pads were removed by dissection, weighed and used to calculate fat pad mass as a proportion of total mass for each animal. Error bars are SEM of n=6 animals. Statistical significance calculated with Mann-Whitney U test (A, E-G, I) or Sidak's multiple comparison test (H) (*p<0.05).

### DETAILED DESCRIPTION OF THE INVENTION

In the research work leading to the present invention, it was very surprisingly found that the protein ZG16 can bind to Gram positive bacteria via binding to the cell wall peptidoglycan. It was furthermore established that treatment with recombinant ZG16 results in bacterial aggregation and inhibition of mucus penetration, which in turn moves bacteria further away from the host epithelium. Thus, the administration of ZG16 will hinder bacteria from getting into contact with the epithelium, and thereby, diseases triggered by bacteria reaching the epithelial surface may be prevented or treated.

This is a ground-breaking finding, which has the potential to revolutionize the prophylaxis and treatment of gastrointestinal disorders.

The intestine contains 10¹³-10¹⁴ bacteria that need to be maintained in a balanced homeostatic relationship with the host. An important mechanism for this is the anti-bacterial peptides and proteins produced by Paneth cells and other cells of the epithelium. However, the first system that luminal bacteria will encounter is the intestinal mucus which is mainly formed from gel-forming mucins produced by goblet cells. Mucins are large, highly glycosylated proteins, and the major intestinal mucin is MUC2. Intestinal mucus is differently organized in the small and large intestine, however, the mucin of both the small and large intestine is composed of MUC2.

The small intestine has a single layer of mucus that is easily removed and penetrable to bacteria and thus does not exclude bacteria from the epithelium. The number of bacteria in the small intestine increases in the distal direction, but overall the number of bacteria in this region is kept at relatively low levels. Rapid small intestinal peristaltic movement of its luminal content, including mucus-trapped bacteria, is a major factor in maintaining a lower bacterial burden. The mucus itself is also important as it firstly lowers the rate of bacterial diffusion towards the epithelium and secondly maintains a gradient of anti-bacterial peptides and proteins that increases in concentration from the lumen to the epithelium. Defects in this system lead to bacterial overgrowth as shown for example in cystic fibrosis where the mucus is attached to the epithelium and is therefore not efficiently transported by peristalsis. (See e.g. Gustafsson, J. K., et. al. 2012. Bicarbonate and functional CFTR channel is required for proper mucin secretion and link Cystic Fibrosis with its mucus phenotype. J. Exp. Med. 209:1263-1272). This could explain why patients with cystic fibrosis sometimes develop distal intestinal obstruction syndrom (DIOS).

The large intestine has much slower peristaltic movement and harbors an enormous amount of commensal bacteria that must be kept physically separated from the epithelium as accomplished by a two-layered mucus system (Johansson, M. E. V., et. al. 2008. The inner of the two Muc2 mucin dependent mucus layers in colon is devoid of bacteria. Proc. Natl. Acad. Sci. USA 105:15064-15069.). The inner mucus layer is laminated, dense, and attached to the epithelium and acts as a filter to exclude bacteria. This filter function is made possible by the macromolecular structure of the large and glycosylated MUC2 mucin which by its disulfide-bonded polymeric nature forms enormous large net-like sheets. At a distance from the epithelium of about 50 μm in mouse and 200 μm in humans, the inner mucus layer is released from its attachment and slowly starts to expand in a process controlled by the host. The same mucin, MUC2, thus behaves differently in the small and large intestine: the loose, unattached mucus layer of the small intestine has similar properties to the outer colon mucus layer. The expanded outer mucus layer is penetrable and is colonized by bacteria. These bacteria are for most of the time beneficial for the host as they ferment endo- and exogenous saccharides to short-chain fatty acids used as nutrients by the host. However, in case of defects in the inner mucus layer, bacteria may penetrate and reach the epithelium. Such an increased load of bacteria close to the epithelium may trigger a bacterial infection or an immune system response, such as an inflammation. It has been shown that defects in the inner mucus layer may lead to e.g. ulcerative colitis. (See e.g. Johansson, M. E. V., et. al. 2014. Bacteria penetrate the normally impenetrable inner colon mucus layer in both murine colitis models and in patients with ulcerative colitis. Gut 213:281-291.)

Besides the MUC2 mucin, proteomic studies of the colonic mucus have revealed additional molecules which are highly abundant in the mucus. One of these is ZG16. However, up until now, there has not been presented any physiologically relevant function of ZG16 in literature. The finding of the present invention, i.e. that ZG16 binds to peptidoglycan, resulting in bacterial aggregation and inhibition of mucus penetration, therefore for the first time presents a physiologically relevant function of ZG16. This in turn provides for the principle of using ZG16 for pharmaceutical purposes. In particular, by utilizing the ability of ZG16 to keep bacteria further away from the host epithelium, it may be used to prevent, treat or alleviate the symptoms of disorders and diseases caused e.g. by bacteria reaching the epithelial surface.

Without wishing to be bound by theory, a proposed mechanism of action of the administration of ZG16 is as follows:

The administered ZG16 binds to bacteria in order to form aggregates of bacteria. ZG16 aggregated bacteria are larger in volume than a single bacterium. This results in the aggregates being size-excluded to travel further through the mucus, such as defect mucus, in the direction of the epithelium. Since the mucus is constantly renewed and secreted from the goblet cells of the epithelium, the aggregated bacteria are trapped in the mucus and travel with the mucus in the direction of the lumen of the gastrointestinal tract. By ZG16 aggregation of the bacteria, the bacteria become less motile compared to a single bacterium, and hence less prone to come close to the epithelium and to translocate into the epithelial cells or further into the tissue surrounding the gastrointestinal tract. Thereby, diseases and disorder caused by bacteria coming into contact with the epithelium may be prevented, alleviated or cured.

In the experimentation underlying the present invention, it has been shown that ZG16 binds to Gram positive bacteria via binding to the cell wall peptidoglycan. However, it may be contemplated that ZG16 also has the ability to bind to Gram negative bacteria, since Gram negative bacteria also have peptidoglycan in the cell wall, albeit to a significantly lesser extent than Gram positive bacteria.

As outlined above, previous studies have shown that:
1. In cystic fibrosis, the small intestine mucus layer is not freely movable, leading to a bacterial overgrowth, which might explain the intestinal symptoms of this disease.
2. A defective functioning of the inner mucus layer of the colon might be a pathophysiological mechanism for colitis and infectious diseases.

Furthermore, as will be evidenced in the examples below, the present invention provides evidence that a penetrable mucus allows more bacteria to translocate into systemic tissues, something that may triggers a systemic inflammation and obesity.

Thus, there is thorough basis for establishing that bacterial contact with epithelial tissue is undesired, as it triggers various kinds of disease states through different mechanisms. The new opportunity presented by the present invention, i.e. to prevent bacteria from getting into contact with epithelium by administering ZG16, is therefore a plausible treatment for a vast number of diseases which are associated with an undesired contact between bacteria and epithelium.

Within the meaning of the present invention, the term “protein based on zymogen granule 16 protein (ZG16)” refers to, and encompasses:
all types of naturally occurring ZG16, such as human and/or animal ZG16 which may naturally be found in the human or animal body, such as in the gastrointestinal tract;
all types of recombinant ZG16 based on the amino acid sequences of naturally occurring human and/or animal ZG16;
any functionally equivalent analogs, homologs and/or derivatives, including modified versions and/or fragments, of the above-described naturally occurring human and/or animal, and/or recombinant, ZG16 proteins. In particular, a recombinant protein based on ZG16 may include various affinity tags added to the protein in order to facilitate production thereof. A non-exhaustive list of examples of such affinity tags are:
   polyhistidine-tags, such as His X 4-6;
   myc-tags having the sequence corresponding to: Glu-Gln-Lys-Leu-Ile-Ser-Glu-Glu-Asp-Leu (EQKLISEEDL);
   fc-tags, such as a fc gamma sequence having its origin from mouse with the corresponding protein sequence:

VYPTSCSRCS RGSDDDDKAE PRGPTIKPCP
PCKCPAPNLL GGPSVFIFPP KIKDVLMISL
SPIVTCVVVD VSEDDPDVQI SWFVNNVEVH
TAQTQTHRED YNSTLRVVSA LPIQHQDWMS
GKEFKCKVNN KDLPAPIERT ISKPKGSVRA
PQVYVLPPPE EEMTKKQVTL TCMVTDFMPE
DIYVEWTNNG KTELNYKNTE PVLDSDGSYF
MYSKLRVEKK NWVERNSYSC SVVHEGLHNH
HTTKSFSRTP GKRTYHHHHH HARKLTR
(fc gamma sequences from other types
of animals may likewise be used).

It is to be understood that the surprising effects of the present invention may be achieved by using various versions of ZG16. ZG16 is in itself a known protein, and its amino acid sequence in humans and various animal species is freely available in renowned reference sequence databases, such as UniProt (www.uniprot.org) and NCBI (www.ncbi.nlm.nih.gov).

As will be further explained and illustrated below, the ZG16 amino acid sequence is highly conserved between species, and it is therefore expected that animal ZG16 showing a high similarity with human ZG16 will also possess the desired properties of aggregating bacteria and inhibiting mucus penetration. Furthermore, it is expected that minor variations in these human and animal amino acid sequences, such as a limited number of substitutions, additions and/or deletions, may be contamplated as long as the desired functionality of the protein in maintained.

In embodiments of the invention, the protein based on ZG16 comprises the sequence motif defined by SEQ ID NO. 1 (optionally excluding the amino acids in positions 1-20, or 1-16, see further below). The sequence motif of SEQ ID NO. 1 has been prepared based on the amino acid sequence of human ZG16 and the amino acid sequences of ZG16 for 19 different animal species showing a sequence identity of 80% or more with the human ZG16 amino acid sequence, see Table 1.

The sequences underlying all sequence identity analyses in the present disclosure are the reference sequences available at UniProt at the date of sequence identity analysis (Oct. 14, 2014). All the compared sequences are also included in the Sequence Listing, for relevant denotation, see Table 1.

SEQ ID NO. 1 contains 167 amino acid positions, of which 89 harbour pre-defined amino acids, i.e. these 89 positions contain amino acids which are completely conserved between the human ZG16 and ZG16 of all the 19 animal species. In view of the conservation of these amino acids in 89 amino acid positions among all the analysed species, it can be concluded that these 89 amino acids are likely of particular relevance for the functionality of the protein. The amino acids of the remaining 78 positions (denoted by “Xaa” in the sequence listing) may vary, and a preferred, but non-exhaustive, list of variations for each position are described in Table 2 by reference to one letter codes. Table 3 shows a list of the three letter code corresponding to each one letter code.

In embodiments of the invention, the protein based on ZG16 comprises the sequence motif defined by SEQ ID NO. 2 (optionally excluding the amino acids in positions 1-20, or 1-16, see further below). The sequence motif of SEQ ID NO. 2 has been prepared based on the amino acid sequence of human ZG16 and the amino acid sequences of ZG16 for 15 different animal species showing a sequence identity of 84% or more with the human ZG16 amino acid sequence, see Table 1.

SEQ ID NO. 2 contains 167 amino acid positions, of which 104 harbour pre-defined amino acids, i.e. these 104 positions contain amino acids which are completely conserved between the human ZG16 and ZG16 of all the 15 animal species. The amino acids of the remaining 63 positions (denoted by “Xaa” in the sequence listing) may vary, and a preferred, but non-exhaustive, list of variations for each position are described in Table 2 by reference to one letter codes.

In embodiments of the invention, the protein based on ZG16 comprises the sequence motif defined by SEQ ID NO. 3 (optionally excluding the amino acids in positions 1-20, or 1-16, see further below). The sequence motif of SEQ ID NO. 3 has been prepared based on the amino acid sequence of human ZG16 and the amino acid sequences of ZG16 for 6 different animal species showing a sequence identity of 90% or more with the human ZG16 amino acid sequence, see Table 1.

SEQ ID NO. 3 contains 167 amino acid positions, of which 138 harbour pre-defined amino acids, i.e. these 138 positions contain amino acids which are completely conserved between the human ZG16 and ZG16 of all the 6 animal species. The amino acids of the remaining 29 positions (denoted by “Xaa” in the sequence listing) may vary, and a preferred, but non-exhaustive, list of variations for each position are described in Table 2 by reference to one letter codes.

Although the amino acid may vary in some of the amino acid positions of each of the sequence motifs of SEQ ID NO:s 1-3, it is possible, for each position, to identify an amino acid being the most frequently occuring among all species. By identifying the most frequently occuring amino acid in each of the 79 variable amino acid positions, a consensus sequence may be established. This consensus sequence is described in SEQ ID NO. 4.

TABLE 1
Amino acid sequence identity of human ZG16 (having
SEQ ID NO. 6; UniProt entry O60844) with ZG16
from different animal species.

| | | | | Amino acid |
|---|---|---|---|---|
| | | UniProt | | sequence |
| Animal species | SEQ ID NO. | entry | Length | identity (%) |
| Rattus norvegicus | 9 | Q8CJD3 | 167 | 85 |
| Mus musculus | 10 | Q8K0C5 | 167 | 84 |
| Bos taurus | 11 | E1BFY4 | 167 | 80 |
| Pan troglodytes | 12 | H2QAV3 | 167 | 99 |
| Sarcophilus harrisii | 13 | G3W5G8 | 165 | 81 |
| Felis catus | 14 | M3WK52 | 167 | 85 |
| Equus caballus | 15 | F7BB58 | 166 | 84 |
| Cavia porcellus | 16 | H0VU97 | 167 | 80 |
| Macaca mulatta | 17 | F6YBH9 | 167 | 94 |
| Canis familiaris | 18 | E2RS34 | 167 | 89 |
| Callithrix jacchus | 19 | F6X6K2 | 167 | 89 |
| Pongo abelii | 20 | H2NRX6 | 167 | 96 |
| Gorilla gorilla gorilla | 21 | G3QR43 | 167 | 99 |
| Otolemur garnettii | 22 | H0XPT3 | 167 | 89 |
| Mustela putorius furo | 23 | M3YLZ5 | 167 | 85 |
| Ovis aries | 24 | W5P6K7 | 167 | 80 |
| Loxodonta africana | 25 | G3TM34 | 167 | 85 |
| Nomascus | 26 | G1R7J5 | 167 | 93 |
| leucogenys | | | | |
| Spermophilus | 27 | I3LYM1 | 161 | 90 |
| tridecemlineatus | | | | |

TABLE 2

| Amino | Possible | Possible | Possible | Consensus |
|---|---|---|---|---|
| acid | variations, SEQ | variations, SEQ | variations, SEQ | sequence, |
| position | ID NO 1 | ID NO 2 | ID NO 3 | SEQ ID NO 4 |
| X1 | M | M | M | M |
| X2 | L, W | L | L | L |
| X3 | T, A, I | T, A, I | T, I | T |
| X4 | V, I, A, L, T | V, I | V, I | V |
| X5 | A, V, T | A, T | A, T | A |
| X6 | L, I | L, I | L, I | L |
| X7 | L, I | L, I | L | L |
| X8 | A, V | A, V | A | A |
| X9 | L | L | L | L |
| X10 | L, F, V | L, V | L | L |
| X11 | C | C | C | C |
| X12 | A, V | A | A | A |
| X13 | S, F | S | S | S |
| X14 | A, T, V | A, V | A, V | A |
| X15 | S, E | S | S | S |
| X16 | G, S, A, V | G, A, V | G, A | A |
| X17 | N, T, S, K, D, none | N, S, K, D | N, D | N |
| X18 | A, S, E, none | A, S | A | A |
| X19 | I, V, S | I, V | I, V | I |
| X20 | Q | Q | Q | Q |
| X21 | A, S, P, V | A, S, V | A | A |
| X22 | R, K, Q | R, Q | R | R |
| X23 | S, T, A | S, T, A | S | S |
| X24 | S | S | S | S |
| X25 | S | S | S | S |
| X26 | Y | Y | Y | Y |
| X27 | S, N | S, N | S, N | S |
| X28 | G | G | G | G |
| X29 | E, D | E, D | E | E |
| X30 | Y, F | Y, F | Y | Y |
| X31 | G | G | G | G |
| X32 | G | G | G | G |
| X33 | G, K, D, R, S | G, K, D, R, S | G | G |
| X34 | G | G | G | G |
| X35 | G | G | G | G |
| X36 | K, E, G, Q | K, E, G, Q | K, Q | K |
| X37 | R | R | R | R |
| X38 | F | F | F | F |
| X39 | S | S | S | S |
| X40 | H, Q | H | H | H |
| X41 | S | S | S | S |
| X42 | G | G | G | G |
| X43 | N, Y | N, Y | N | N |
| X44 | Q | Q | Q | Q |
| X45 | L | L | L | L |
| X46 | D, E | D, E | D | D |
| X47 | G | G | G | G |
| X48 | P | P | P | P |
| X49 | I | I | I | I |
| X50 | T | T | T | T |
| X51 | A | A | A | A |
| X52 | L, I, F | L, I, F | L | L |
| X53 | R | R | R | R |
| X54 | V, I | V, I | V | V |
| X55 | R | R | R | R |
| X56 | V, I | V, I | V | V |
| X57 | N, S | N, S | N, S | N |
| X58 | T, R, N, G, K, S | T, R, N, G, K | T, R, N, K | R |
| X59 | Y, none | Y, none | Y | Y |
| X60 | Y | Y | Y | Y |
| X61 | I | I | I | I |
| X62 | V, I | V, I | V | V |
| X63 | G | G | G | G |
| X64 | L | L | L | L |
| X65 | Q | Q | Q | Q |
| X66 | V | V | V | V |
| X67 | R | R | R | R |
| X68 | Y | Y | Y | Y |
| X69 | G | G | G | G |
| X70 | K, T | K, T | K, T | K |
| X71 | V, E | V | V | V |
| X72 | W | W | W | W |
| X73 | S | S | S | S |
| X74 | D, N, A | D, N, A | D | D |
| X75 | Y, H, F | Y, H | Y | Y |
| X76 | V | V | V | V |
| X77 | G | G | G | G |
| X78 | G | G | G | G |
| X79 | R, N, T, S, K | R, N, T, S, K | R, S, K | T |
| X80 | N, Q, S, G, L | N, Q, S, G, L | N, S | S |
| X81 | G | G | G | G |
| X82 | D, N | D, N | D | D |
| X83 | L | L | L | L |
| X84 | E, D | E | E | E |
| X85 | E | E | E | E |
| X86 | I | I | I | I |
| X87 | F | F | F | F |
| X88 | L | L | L | L |
| X89 | H, Y | H | H | H |
| X90 | P, S | P | P | P |
| X91 | G | G | G | G |
| X92 | E | E | E | E |
| X93 | S | S | S | S |
| X94 | V, I | V | V | V |
| X95 | I, V | I | I | I |
| X96 | Q | Q | Q | Q |
| X97 | V | V | V | V |
| X98 | S | S | S | S |
| X99 | G | G | G | G |
| X100 | K | K | K | K |
| X101 | Y | Y | Y | Y |
| X102 | K, E, S | K, E | K | K |
| X103 | W, S, T, Y, K, G, | W, S, Y, K, G, R, | W, G, S, N | S |
| | R, N, F | N, F | | |
| X104 | Y | Y | Y | Y |
| X105 | L, V | L, V | L | L |
| X106 | K, R | K, R | K, R | R |
| X107 | K, Q | K, Q | K | K |
| X108 | L, M, V | L, M, V | L, V | L |
| X109 | L, I, V | L, I, V | L, V | V |
| X110 | F | F | F | F |
| X111 | V | V | V | V |
| X112 | T | T | T | T |
| X113 | D | D | D | D |
| X114 | K | K | K | K |
| X115 | G, F | G | G | G |
| X116 | R | R | R | R |
| X117 | Y, F | Y | Y | Y |
| X118 | L | L | L | L |
| X119 | S, P, A | S, P, A | S, P, A | P |
| X120 | F | F | F | F |
| X121 | G | G | G | G |
| X122 | K, T | K, T | K | K |
| X123 | D, A | D, A | D | D |
| X124 | S, T, I, K | S, T, I | S, T | T |
| X125 | G | G | G | G |
| X126 | T | T | T | T |
| X127 | S | S | S | S |
| X128 | F | F | F | F |
| X129 | N, S | N, S | N | N |
| X130 | A | A | A | A |
| X131 | V, A, L | V, A | V | V |
| X132 | P | P | P | P |
| X133 | L | L | L | L |
| X134 | H, Y | H, Y | H | H |
| X135 | P | P | P | P |
| X136 | N | N | N | N |
| X137 | T | T | T | T |
| X138 | V | V | V | V |
| X139 | L | L | L | L |
| X140 | R | R | R | R |
| X141 | F | F | F | F |
| X142 | I, F | I | I | I |
| X143 | S | S | S | S |
| X144 | G | G | G | G |
| X145 | R | R | R | R |
| X146 | S, A | S, A | S, A | S |
| X147 | G, S | G, S | G | G |
| X148 | S, A, I | S, A, I | S | S |
| X149 | L, A, F, V | L, A, F, V | L, V | L |
| X150 | I | I | I | I |
| X151 | D, N | D, N | D | D |
| X152 | A, S | A, S | A | A |
| X153 | I | I | I | I |
| X154 | G, S | G, S | G | G |
| X155 | L, F | L | L | L |
| X156 | H | H | H | H |
| X157 | W | W | W | W |
| X158 | D | D | D | D |
| X159 | V, T, L, S | V, T, S | V | V |
| X160 | Y | Y | Y | Y |
| X161 | P | P | P | P |
| X162 | S, NONE | S, NONE | S, NONE | S |
| X163 | S, H, D, E, N, I, T, | S, H, D, N, I, T, V, | S, I, NONE | D |
| | V, NONE | NONE | | |
| X164 | C, Y, NONE | C, NONE | C, NONE | C |
| X165 | S, N, E, G, NONE | S, N, G, NONE | S, NONE | S |
| X166 | R, T, S, K, NONE | R, T, S, K, NONE | R, S, K, NONE | S |
| X167 | C, NONE | C, NONE | C, NONE | C |

TABLE 3

| Amino acid | Three letter code | One letter code |
|---|---|---|
| alanine | ala | A |
| arginine | arg | R |
| asparagine | asn | N |
| aspartic acid | asp | D |
| asparagine or aspartic acid | asx | B |
| cysteine | cys | C |
| glutamic acid | glu | E |
| glutamine | gln | Q |
| glutamine or glutamic acid | glx | Z |
| glycine | gly | G |
| histidine | his | H |
| isoleucine | ile | I |
| leucine | leu | L |
| lysine | lys | K |
| methionine | met | M |
| phenylalanine | phe | F |
| proline | pro | P |
| serine | ser | S |
| threonine | thr | T |
| tryptophan | trp | W |
| tyrosine | tyr | Y |
| valine | val | V |

In preferred embodiments of the invention, the protein based on ZG16 is defined by SEQ ID NO:s 5-8 (optionally excluding the amino acids in positions 1-20, or 1-16), corresponding to the amino acid sequence for human ZG16. It is to be noted that natural variations may exist in the sequence, as illustrated by SEQ ID NO 5. SEQ ID NO:s 6-8 define three equally preferred specific variants of human ZG16: SEQ ID NO 6 corresponds to the human ZG16 amino acid sequence defined by Uniprot entry O60844; SEQ ID NO 7 corresponds to the human amino acid sequence defined by NCBI Reference Sequence no NP_689551.1; SEQ ID NO 8 corresponds to the human amino acid sequence defined by NCBI Reference Sequence no. NP_689551.2.

In embodiments of the invention, the protein based on ZG16 is defined by SEQ ID NO. 9-27 (optionally excluding the amino acids in positions 1-20, or 1-16, as defined by the general sequence motif set forth in SEQ ID NO 1), corresponding to the amino acid sequence for ZG16 of the 19 various animal species listed in Table 1.

In embodiments of the invention, the protein based on ZG16 has a sequence identity of at least 80%, such as at least 84%, at least 90%, or at least 99% with human ZG16 as defined by SEQ ID NO. 6 (optionally excluding the amino acids in positions 1-20, or 1-16).

In all the above-described variants of the the protein based on ZG16, the amino acids in positions 1-20, or 1-16 as defined by the general sequence motif set forth in SEQ ID NO 1 may be omitted (i.e. the amino acids in positions 1-20, or 1-16 may be optionally excluded). The amino acids in positions 1-20, or 1-16 constitute a signal sequence, and as such have no functionality in the resulting protein. Generally, recombinant versions of the protein based on ZG16 do not include the amino acids in positions 1-20, or 1-16 as defined by the general sequence motif set forth in SEQ ID NO 1.

Thus, in embodiments of the invention, the protein based on ZG16 comprises the amino acids in positions 21-167 of any one of SEQ ID NO 1-3 (i.e. amino acids 1-20 omitted), or alternatively the protein based on ZG16 comprises the amino acids in positions 17-167 of any one of SEQ ID NO 1-3 (i.e. amino acids 1-16 omitted).

In other embodiments, the protein based on ZG16 comprises the amino acids in positions 21-167 of any one of SEQ ID NO 5-8 (i.e. amino acids 1-20 omitted), or alternatively the protein based on ZG16 comprises the amino acids in positions 17-167 of any one of SEQ ID NO 5-8 (i.e. amino acids 1-16 omitted).

In other embodiments, the protein based on ZG16 comprises the amino acids in positions 21-167 of any one of SEQ ID NO 9-12, 14 or 16-26 (i.e. amino acids 1-20 omitted), or alternatively the protein based on ZG16 comprises the amino acids in positions 17-167 of any one of SEQ ID NO 9-12, 14 or 16-26 (i.e. amino acids 1-16 omitted).

The protein based on ZG16 which is defined by SEQ ID NO 13 corresponds to a version of the general sequence motif SEQ ID NO 1 wherein amino acids 17 and 18 are NONE. Thus, the protein based on ZG16 which is defined by SEQ ID NO 13 and lacking amino acids 1-16 or 1-20 (as defined by the general sequence motif set forth in SEQ ID NO 1) comprises the amino acids in positions 17-165 and 19-165 of SEQ ID NO 13.

The protein based on ZG16 which is defined by SEQ ID NO 15 corresponds to a version of the general sequence motif SEQ ID NO 1 wherein amino acid 59 is NONE. Thus, the protein based on ZG16 which is defined by SEQ ID NO 15 and lacking amino acids 1-16 or 1-20 (as defined by the general sequence motif set forth in SEQ ID NO 1) comprises the amino acids in positions 17-166 and 21-166 of SEQ ID NO 15.

The protein based on ZG16 which is defined by SEQ ID NO 27 corresponds to a version of the general sequence motif SEQ ID NO 1 wherein amino acids 162-167 are NONE. Thus, the protein based on ZG16 which is defined by SEQ ID NO 27 and lacking amino acids 1-16 or 1-20 (as defined by the general sequence motif set forth in SEQ ID NO 1) comprises the amino acids in positions 17-161 and 21-161 of SEQ ID NO 27.

A recombinant protein based on ZG16 may be produced in conventional ways, well-known by persons skilled in the art.

Examples of diseases that may be treated and/or prevented by in accordance with the present invention are:
Gastrointestinal diseases, such as Irritable Bowel Syndrome (IBS), or gastrointestinal inflammatory diseases. Examples of gastrointestinal inflammatory diseases are systemic inflammatory diseases, or Inflammatory Bowel Disease (IBD), such as Crohn's disease or colitis, such as ulcerative colitis. Also contemplated are gastrointestinal diseases being intestinal symptoms of cystic fibrosis, e.g. DIOS.
Bacterial infections.
Disorders related to the metabolic syndrome, e.g. obesity and/or diabetes, such as type 2 diabetes.

In particular, ZG16 treatment of the intestine can limit the penetration of bacteria into the systemic circulation, systemic inflammation, obesity and increased fat accumulation. These findings are supported by observations in mice lacking Zg16.

By “prevention and/or treatment of a disorder” is meant any treatment in order to cure or alleviate the symptoms of any of the herein described disorders related to a bacterial imbalance in the gastrointestinal tract, or any treatment to prevent the development of such a disorder.

The peptides based on ZG16 may either be used as they are or be included in a pharmaceutical composition. Such a pharmaceutical composition may also comprise substances used to facilitate the production or administration of the pharmaceutical composition. Such substances are well known to persons skilled in the art and may for example be pharmaceutically acceptable adjuvants, carriers and preservatives. It is also possible to include the peptides based on ZG16, in an effective amount, in any kind of food or beverage.

The pharmaceutical composition may be formulated e.g. for oral, nasal, rectal, topical, intravenous, intraartery, intracavitary, intramuscular, subcutaneous, or transdermal administration, and may e.g. be in the form of a tablet, a capsule, a suppository, a powder, a paste, a solution, a cream, a gel, and/or an administration unit comprising bacteria having the ability to produce and secrete a protein based on ZG16.

In one embodiment of the invention, the gene encoding ZG16 can be inserted into any bacteria having the ability to produce and secrete ZG16. This bacteria can then be used as a medicament and given e.g. orally or rectally.

By the term “pharmaceutically effective amount of a protein based on ZG16” is herein meant an amount of a protein based on ZG16 which is effective to keep bacteria at a distance from the epithelium of the gastrointestinal tract, such as to bind to and aggregate bacteria, such as Gram positive bacteria. In an embodiment, the pharmaceutical composition comprising ZG16 is administered in a concentration within the range of from 1 μg to 100 g.

The term “patient” as used herein encompasses human or animals in need of treatment and/or prevention of a disorder related to a bacterial imbalance in the gastrointestinal tract.

The proteins based on ZG16 may either be used alone, or in combination with conventional therapy.

By the term “bacterial imbalance”, is herein meant an undesired increased load of bacteria close to the epithelium of the gastrointestinal tract and/or penetration of undesired bacteria into an epithelial cell of the epithelium of the gastrointestinal tract and/or penetration of undesired bacteria through the epithelial cell of the gastrointestinal tract into neighbouring tissue of a host. Such a bacterial imbalance may cause a bacterial infection and/or an inflammation.

The invention will now be further explained in the following examples. These examples are only intended to illustrate the invention and should in no way be considered to limit the scope of the invention. Throughout the examples, the term “ZG16”, relates to ZG16 of human origin having the sequence defined by SEQ ID NO. 7.

### EXAMPLES

### Materials and Methods

### Generation of Expression Plasmids

Construction of the pS-ZG16-IgG expression plasmid has been described previously (Rodriguez-Pineiro, A. M., et. al. 2013. Studies of mucus in mouse stomach, small intestine, and colon. II. Gastrointestinal mucus proteome reveals Muc2 and Muc5ac accompanied by a set of core proteins. Am. J. Physiol. Gastroint. Liver Physiol. 305:G348-G356.). For the pcDNA3.1-ZG16 plasmid, total RNA was extracted from the colon adenocarcinoma cell-line LS-174T with RNeasy Mini Kit (Qiagen). With total RNA as template the entire open reading frame of ZG16 was reversed transcribed and amplified with SuperScript One-Step RT-PCR with Platinum Taq (Life Technologies) and inserted into the pcDNA3.1-V5/His-TOPO vector (Life Technologies) to produce the vector pcDNA3.1-ZG16-V5/His. The C-terminal tag was removed by introducing a stop codon after the open reading frame of ZG16 using QuikChange Site-Directed Mutagenesis Kit (Stratagene). Positive clones were controlled by nucleotide sequencing.

### Purification of Recombinant Protein

Spent media from CHO-K1 cells transfected with pS-ZG16-IgG was collected, centrifuged at 4,000 g for 5 min and passed through a 0.22 μm filter to remove cellular debris. Filtered media was then diluted 1:1 with Protein-G binding buffer (20 mM Sodium Phosphate buffer, pH 7.0) and applied to a 5 ml Protein-G column (GE Healthcare). Bound protein was eluted with 0.1 M Glycine-HCl, pH 2.7, and pH in the eluted fractions adjusted to pH 7.0 with 1 M Tris-HCl pH 9.0 buffer. Fractions containing ZG16-IgG was pooled and concentrated using Vivaspin 6, MWCO 10,000 (Sartorius) spin columns. Protein amount after concentration was determined with BCA protein assay (Pierce).

Purification of untagged ZG16 was performed as follows. Spent media from CHO-S cells expressing ZG16 was collected. Media was diluted 1:1 in 50 mM Hepes, pH 8.0 and separated from impurities using ion-exchange chromatography on a ÄKTA Purifer system (GE Healthcare). ZG16 was loaded onto a MONO S 5/5 column equilibrated in 50 mM HEPES pH 8.0, and it was eluted (around 400 mM NaCl with a linear gradient of 0-1.0 M NaCl in the same buffer. ZG16 containing fractions were pooled and desalted with PD-10 column (GE Healthcare). Desalted protein was lyophilized, dissolved in PBS and concentration determined with BCA protein assay (Pierce).

### Trypsin Resistance of ZG16

10 μl trypsin (Lonza) were added to 2 μg purified ZG16 with the additional amino acids VYPTSCSRCSRGSDDDDK volume adjusted to a final volume of 20 μl. The samples were incubated at 37° C. for 2 min and 5 min respectively. Trypsin inactivation was performed with the addition of 20 μl Laemmli sample buffer with 200 mM dithiothreitol (DTT). Samples ran on continuous 18% Laemmli gel. Band from the gel were cut out, in-gel digested with trypsin and analyzed with LC-MS/MS.

### Binding to Peptidoglycan

Binding experiments were performed as a Dissociation-Enhanced Lanthanide Fluorescent Immunoassay (DELFIA) based method in V-shaped NUNC microtiter plates as follows. Insoluble peptidoglycan from Bacillus subtilis (Sigma), Lactobacillus jensenii (CCUG 35572T), or Eschericia coli (K12)bacteria were incubated with different concentrations of ZG16-IgG or MUC1-IgG at +4° C. overnight. Peptidoglycan or bacteria were pelleted by centrifugation at 1,200 g for 5 min. Supernatant discarded and unspecific binding prevented by incubating the plates for 1 h at RT in DELFIA Blocking solution. Plate centrifuged and the blocking solution aspirated. Europium labeled donkey anti-mouse antibody diluted 1:200 in DELFIA Assay solution added to the wells. Each well was washed 5 times with DELFIA Wash solution before 200 μl DELFIA Enhancement solution was added. Fluorescent signal was read in VICTOR2 plate reader (Wallac). Each assay was performed in triplicates. For inhibition experiments recombinant protein was pre-incubated with MurNAc (Sigma) before addition to the wells.

### Bacterial Culture

Escherichia coli, Enterococcus faecalis and Bacillus subtilis were routinely cultured from frozen glycerol stocks in LB or BHI at 37° C. for 15 h. Lactobacillus jensenii was grown at 37° C. for 15 mh in MRS-broth (Merck). Cultures were then diluted 1:100 in fresh LB, BHI, or MRS and grown to the required cell density for each experiment.

### Bactericidal Assays

Lactobacillus jensenii culture was diluted 1:100 in fresh MRS-broth and 100 μl transferred to a 96-well plate (Falcon). 100 μl spent cell culture media containing recombinant ZG16 from transfected CHO-K1 cells added to the wells. Absorbance (560 nm) was read at 0, 2, 4, 6, 8 and 24 h in a Victor plate reader (Wallac) in triplicates. The radial diffusion assay was performed with untagged purified ZG16 and Bacillus subtilis (CCUG 163T) as described (Schroeder 2011. Reduction of disulphide bonds unmasks potent antimicrobial activity of human BGR-defensin 1. Nature 469, 419-423).

### Bacterial Aggregation Assay

Bacterial cultures were grown to OD₆₀₀ 0.1 in LB. 1 mL of culture was centrifuged at 5,000 g for 5 min and cells were washed with PBS. Washed cells were re-centrifuged and resuspended in PBS with or without 10 μg rZG16 and incubated for 30 min at room temperature. Cell suspensions were transferred to microscope slides and fixed by heating. Slides were submerged in 10 μM Syto9 nucleic acid stain (Life Technologies) for 30 min then washed twice in PBS. Stained cells were imaged using a confocal microscope (Zeiss LSM 700) using a 488 nm laser and a Plan-Apochromat 20x/0.8 M27 objective lens. Multiple 2.5 mm² tile scans were acquired from each slide using Zen software (Zeiss). Zen files were exported to Velocity (Perkin Elmer) and the areas of individual Syto9 stained objects were quantified.

Bacillus subtilis Motility Assay

Motility agar plates were prepared using BHI growth medium containing 0.3% (w/v) agar. After the molten agar had cooled, 50 μL of PBS with 20 μg BSA or 20 μg rZG16 was added to the center of plate and allowed to diffuse into the agar. 2 μL OD600 0.5 Bacillus subtilis was inoculated into the center of the plate and plates were then incubated for 6 h at 37° C. In order visualize bacteria in the agar, plates were first washed with PBS to remove bacteria at the agar surface then flooded with 10 μM Syto9 and incubated at room temperature for 30 min. Plates were then washed with PBS and stained cells within the agar were imaged using a confocal microscope (Zeiss LSM 700) using a 488 nm laser and a Plan-Apochromat 20x/0.8 M27 objective lens. Tile scans (20 mm×0.64 mm) were acquired using Zen and exported into Velocity (Perkin Elmer) where fluorescence intensity data from each 1 mm section was extracted. Fluorescence data for each section was normalized to total fluorescence for the whole tile scan and normalized data was used to compare bacterial distribution in the agar between treatment groups.

### Animals

The Zg16−/− mice was obtained from Taconic (Accession number: TF0327), which was generated by deletion of all three Zg16 exons by the insertion of a resistance gene. Mice were backcrossed >10 generations into a C57BL/6 background. WT C57BL/6 mice used for backcross was used as control animals. All animal experiments were performed according to local ethics committee guidelines. Animals were weighed to determine total mass, after which abdominal fat pads were removed by dissection, weighed and used to calculate fat pad mass as a proportion of total mass for each animal. Error bars are SEM of n=6 animals, statistical significance calculated by Mann-Whitney test

### Mucus Measurements

Mucus measurements on Zg16−/− (n=6) and WT (n=9) were performed as previously described (Gustafsson, J. K., et. al.. 2012. An ex vivo method for studying mucus formation, properties and thickness in human colonic biopsies and mouse small and large intestinal explants. Am. J. Physiol. Gastrointest. Liver. Physiol. 302:G430-G438.). Short, distal colon tissue was mounted in a horizontal perfusion chamber, charcoal added to visualize the mucus and the distance between the epithelial cell surface to the charcoal measured with a micropipette. The mucus growth was monitored for 60 min. For one of the two tissue samples taken from each animal 1 mM carbachol was added to the serosal side after 30 min.

### Mucus Penetrability Measurements

The penetrability of the distal colon mucus was measured as previously described (Gustafsson, J. K., et. al. 2012. Supra.). Briefly, distal colon explants was mounted in a perfusion chamber as for the mucus thickness experiments. Explants were incubated for 20 min before and a suspension of 0.5 μm (red), 1 μm (far red), and 2 μm (green) fluorescent beads (FluoSpheres, Life Technologies) was added. The beads were allowed to sediment for 40 min. Z-stacks using a LSM700 microscope (Carl Zeiss) were taken and the distribution of the beads in the mucus analyzed. Acquired results were analyzed in Velocity (Perkin Elmer).

### Immunohistochemistry

Zg16−/− and WT controls were euthanized by isoflurane and cervical dislocation. Distal colon with fecal material was fixed in Carnoy solution (MethaCarn) to preserve the mucus (Johansson, M. E. V., et. al. 2008. The inner of the two Muc2 mucin dependent mucus layers in colon is devoid of bacteria. Proc. Natl. Acad. Sci. USA 105:15064-15069.). Fixed tissue was paraffin embedded, sectioned, dewaxed, and stained with Haematoxylin-Eosin, Alcian blue/periodate acid Schiff (PAS) or Brown-Brenn. For fluorescent microscopy dewaxed sections was stained with the anti MUC2C3 antiserum (Johansson, M. E. V., et. al. 2008. The inner of the two Muc2 mucin dependent mucus layers in colon is devoid of bacteria. Proc. Natl. Acad. Sci. USA 105:15064-15069) followed by anti-rabbit IgG Alexa 488 (Life Technologies) and with Gram positive LTA mouse monoclonal antibody (Thermo Scientific) followed by anti-mouse IgG1 Alexa 555 (Life Technologies). DNA was counterstained with Hoescht 35258 (Molecular Probes) and mounted with ProLong Gold (Life Technologies). Pictures were obtained using a Nikon E-1000 fluorescent microscope (Nikon).

### Bacterial Ex-Vivo Mucus Penetration

Colonic tissue was mounted in a horizontal perfusion imaging chamber and 2 μm Fluosphere beads were applied apically (as for mucus penetrability assays) in order to visualize the interface between the impenetrable (IM) and penetrable mucus (PM) layers. 1 mL OD₆₀₀ 0.1 bacterial cultures were centrifuged at 5,000 g for 5 min and resuspended in PBS. Cells were labeled using 0.2 μM BacLight Red stain and incubated in the dark at RT for 15 min. Cells were then centrifuged at 5,000 g for 5 min and resuspended in Krebs-mannitol buffer with or without 10 μg rZG16 and incubated at RT for 30 min. Labeled bacteria were added to colonic explant mucus after addition of beads and incubated at 37° C. for 15 min. Bacteria and beads were imaged using a confocal microscope (Zeiss LSM 700) using 488/555 nm lasers and a Plan-Apochromat x20/1.0DIC water objective lens. Confocal z-stacks covering the IM/PM interface were acquired and fluorescence values for beads and labeled bacteria at each focal plane were recorded using Zen software (Zeiss). Fluorescence values for each stack were normalized to total fluorescence and used to determine the distribution of beads and bacteria along the z-axis of the stack. Normalized data from multiple z-stacks were aligned using the IM/PM interface (focal plane with maximum normalized bead fluorescence) to allow comparison of bacterial distribution in the mucus between different treatment groups.

### Visualization and Tracking of Mucus Associated Bacteria

Colonic tissue was mounted in a horizontal perfusion imaging chamber and 2 μm Fluosphere beads were applied apically (as for mucus penetrability assays) in order to visualize the interface between the impenetrable (IM) and penetrable mucus (PM) layers. Mucus associated bacteria and colonic tissues were stained with 10 μM Syto9 in Krebs-mannitol for 30 min at RT. The staining solution was then removed and replaced with fresh Krebs-mannitol buffer and bacteria, tissue and beads were imaged using a confocal microscope (Zeiss LSM 700) using 488/555 nm lasers and a Plan-Apochromat x20/1.0DIC water objective lens. Images and 1 min time series were acquired using Zen software. Time series were exported to Imaris software (Bitplane) in order to track the movement of bacteria within the mucus and beads at the IM/PM interface. The motile capacity (MC) of bacteria and beads was determined by recording the maximum speed of individual detected tracks. Bacteria were classed as motile if their MC exceeded the mean MC of the beads tracked in each time series or were classed as non-motile if their MC equaled or was less than this value.

DNA Extraction from Mucus and Systemic Tissues

Non-adherent mucus was acquired from chamber mounted tissues using a micropipette and tissue adherent mucus from the same area was acquired by microdissection of tissues directly from the chamber. Systemic tissues (mesenteric lymph nodes, live and spleen) were acquired directly by dissection. All samples were transferred to 1 mL TE buffer supplemented with 0.5% SDS (w/v) and 200 μg/mL proteinase K (Qiagen) in Lysing Matrix E tubes (MP Biomedicals). Lysing tubes were incubated at 55° C. for 1 h after which DNA was isolated using mechanical disruption and phenol:chloroform:isoamylalcohol extraction as previously described (Zoetendal, E. G., et. al. 2006. Isolation of DNA from bacterial samples of the human gastrointestinal tract. Nature Protocols 1:870-873.). Extracted DNA was precipitated using isopropanol and 3M sodium acetate. DNA was pelleted by centrifugation at 20 000 g for 20 min and the pellet was washed with 70% ethanol. Finally DNA pellets were rehydrated in 100 μL TE buffer and stored at −20° C. until used. Blank extractions were performed at the same time as all sample DNA extractions in order to control for potential contamination.

16S qPCR Analysis

DNA extracted from mucus and systemic tissues was quantified using a Nanodrop spectrophotometer (Thermo). In order to allow quantification of low copy number bacterial 16S DNA by qPCR the ratio of 16S DNA to total DNA was increased by limited cycle number (LCN) PCRs amplifying the whole 16S gene. 50 μL LCN PCRs were prepared using HotStar Taq Plus PCR Mastermix (Qiagen), 0.2 μM universal forward primer 27F (AGAGTTTGATCMTGGCTCAG) (SEQ ID NO. 28), 0.2 μM universal reverse primer 1492R (CGGTTACCTTGTTACGACTT) (SEQ ID NO. 29) and 500 ng template DNA. Thermocycling conditions were: 1 cycle of 95° C. for 5 min; 16 cycles of 94° C. for 1min, 55° C. for 1 min, 72° C. for 1.5 min; 1 cycle of 72° C. for 10 min. 16S standards (quantified E. coli 16S DNA), contamination controls and no template controls were amplified at the same time as samples. Amplified samples, standards and controls were then analysed by qPCR to determine the total number of 16S copies and the relative proportions of total 16S belonging to three taxonomic groups of bacteria (Bacteroidetes, Firmicutes and Gamma-proteobacteria) as previously described (Bacchetti De Gregoris, T., et. al. 2011. Improvement of phylum- and class-specific primers for real-time PCR quantification of bacterial taxa. J. Microbiol. Methods 86:351-356.). Briefly, 20 μL qPCRs were prepared using 2 μL of LCN PCR amplifications as template, SsoFast EvaGreen qPCR Supermix (Bio-Rad) and 0.3 μM each of one of the following primer pairs targeting the indicated bacterial group: Universal—926F (AAACTCAAAKGAATTGACGG) (SEQ ID NO. 30), 1062R (CTCACRRCACGAGCTGAC) (SEQ ID NO. 31); Bacteroidetes—798cfbF (CRAACAGGATTAGATACCCT) (SEQ ID NO. 32), cfb967R (GGTAAGGTTCCTCGCGTAT) (SEQ ID NO. 33); Firmicutes—928F-Firm (TGAAACTYAAAGGAATTGACG) (SEQ ID NO. 34), 1040FirmR (ACCATGCACCACCTGTC) (SEQ ID NO. 35); Gamma,-proteobacteria—1080 gF (TCGTCAGCTCGTGTYGTGA) (SEQ ID NO. 36), g1202R (CGTAAGGGCCATGATG) (SEQ ID NO. 37). qPCRs were run on a CFX96 instrument (Bio-Rad). Cq values were exported from CFX manager software (Bio-Rad) and calibration curves constructed from 16S standard data. Calibration curves were used to calculate the total original 16S copy number from mucus and systemic tissue sample DNA extractions. 16S data was then normalized to mucus volume or tissue mass in order to allow comparison between different experimental groups.

Bacterial Culture from Systemic Tissues

Systemic tissues were first acquired by dissection and transferred to tubes containing 1 ml sterile PBS. A sterilized 5 mm stainless steel bead was added to each tube and tissues were homogenized for 40 sec using a Fast-Prep 24 instrument (MP Biomedicals). 100 μL of tissue homogenate was spread on BHIS agar plates (BHI agar supplemented with 0.5% w/v yeast extract) and incubated at 37° C. under anaerobic conditions for 48 h after which bacterial growth was assessed and recorded.

### Example 1

### ZG16 Binds Peptidoglycan and G+ Bacteria

As the small protein ZG16 was found to be a major component in colonic mucus and had lectin-like structure, we asked if it was binding the most abundant glycan in bacteria, peptidoglycan.

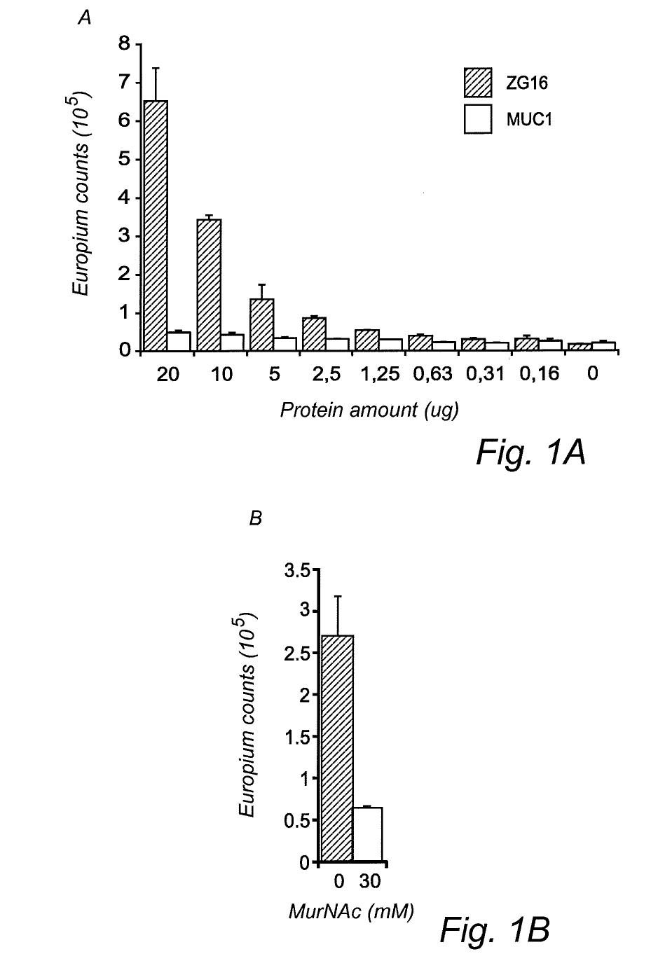

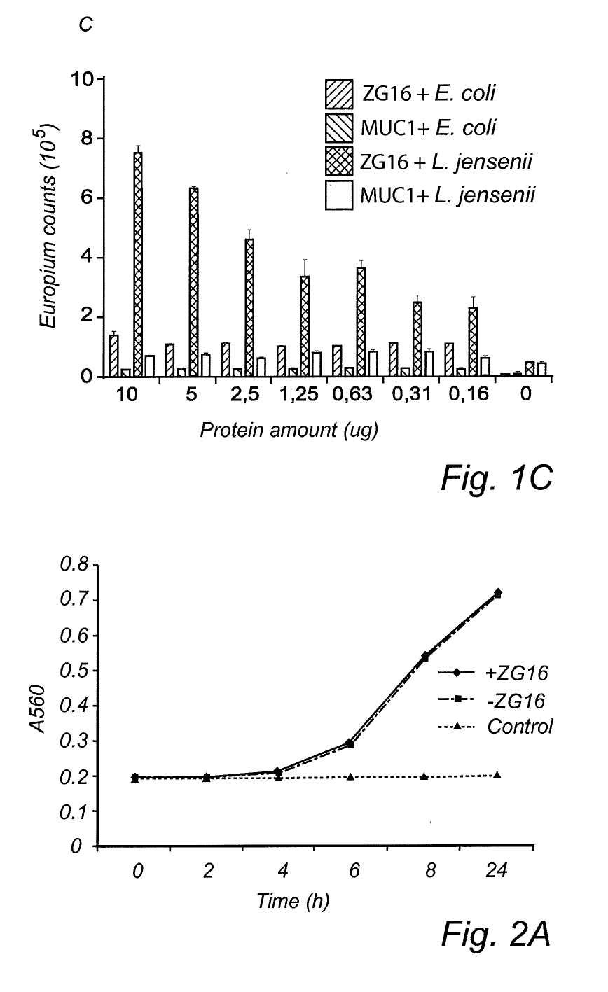

Pure recombinant ZG16-Ig, but not an irrelevant Ig-fusion protein, was found to bind insoluble peptidoglycan in a concentration dependent way (FIG. 1A). This binding was inhibited by one of the sugar building blocks of peptidoglycan, MurNAc, although at a relatively high concentration (FIG. 1B).

Since G+ bacteria has peptidoglycan exposed to the surrounding we further showed that ZG16 could bind to intact G+ bacteria such as Lactobacillus jensenii , but in this case not G− bacteria, i.e. E. coli (FIG. 1C). Similar results as to the insoluble peptidoglycan were observed.

### Example 2

### ZG16 is not Directly Bactericidal, but Aggregate G+ Bacteria

In order to explore if ZG16 affected the viability of bacteria after binding, a bactericidal assay was performed.

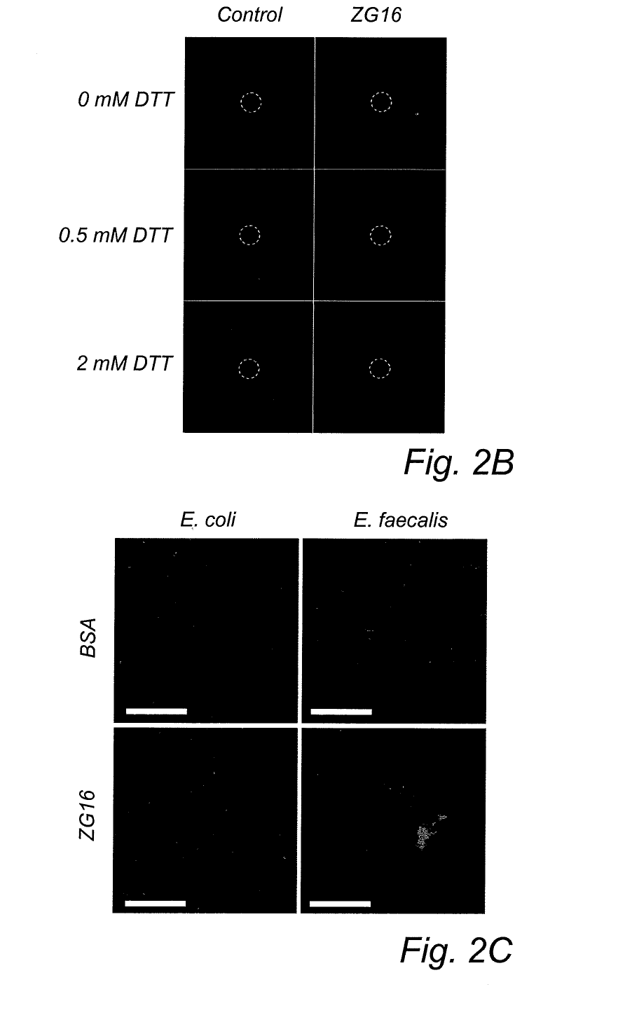

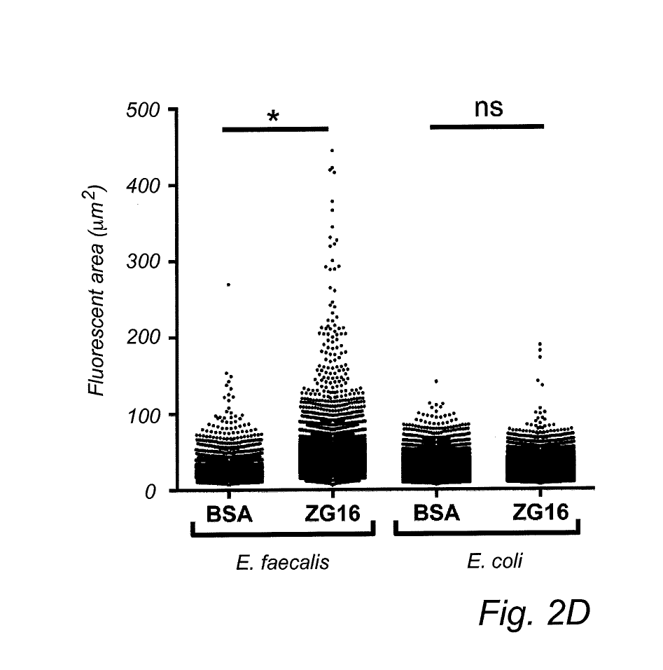

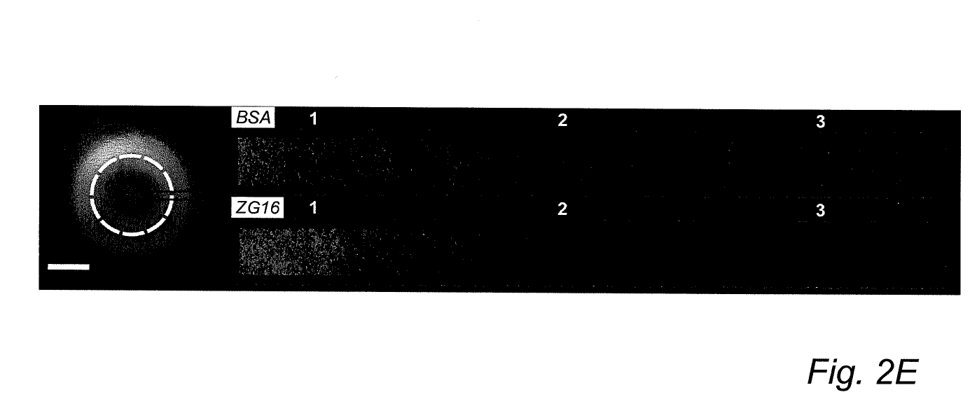

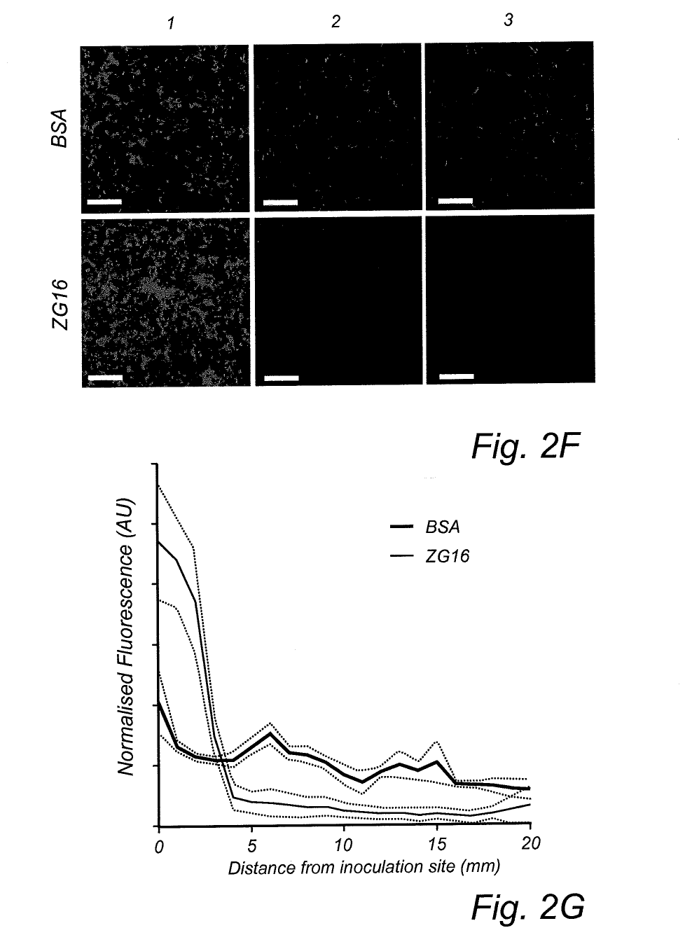

The bacterial growth was monitored by repeated OD⁵⁶⁰ measurements after the addition of recombinant ZG16 to the bacterial growth media (FIG. 2A). The bacterial growth curve was unchanged by the addition of ZG16 (black line) compared to unsupplemented broth (grey). ZG16 contain two C-terminal cysteines proposed to form an intramolecular disulfide bond (Kanagawa, M., et. al. 2011. Crystal structures of human secretory proteins ZG16p and ZG16b reveal a Jacalin-related beta-prism fold. Biochemical and Biophysical Research Communications 404:201-205.) The human β-defensin 1 was shown to only have antimicrobial activity under conditions where its disulfide bonds were reduced (Schroeder 2011. Reduction of disulphide bonds unmasks potent antimicrobial activity of human BGR-defensin 1. Nature 469, 419-423). To test if ZG16 had an antimicrobial activity in the same way, recombinant ZG16 was added to agar plates with increasing concentrations of dithiothreitol (DTT) together with the Gram positive bacteria Bacillus subtilis. This radial diffusion assay did not show any clear zone and thus no effect on the growth of the microbe (FIG. 2B).

Together, the results suggest that ZG16 bind to bacteria, but do not have any antimicrobial activity by itself.

To address if ZG16 had any other effect than just binding G+ bacteria, bacteria treated with ZG16 were spread on microscopy slides, stained and visualized with a fluorescent microscope (FIG. 2C-D). Large bacterial aggregates were observed in contrast to non-treated bacteria. The size of the bacterial aggregates was measured was significantly larger than the control treated with bovine serum albumn (BSA) only (FIG. 2C, bottom right, FIG. 2D). To access the bacterial aggregation in a system that resemble the polymeric mucus network, bacteria were plated on agar plates and the bacterial growth and spread within the plate were observed by confocal microscopy (FIG. 2F-G). Bacteria applied to plates containing rZG16 showed a higher degree of aggregation and decreased capacity to spread in the agar in contrast to BSA treated controls.

Together this data shows that ZG16 does not affect the viability of bacteria after binding, instead ZG16 cause aggregation which limit their ability to penetrate polymeric networks.

### Example 3

### Zg16^{-/-} Mice Mucus Phenotype

To address if ZG16 affect the mucus layer properties, a Zg16 knock out mouse on a C57/BL6 background was studied.

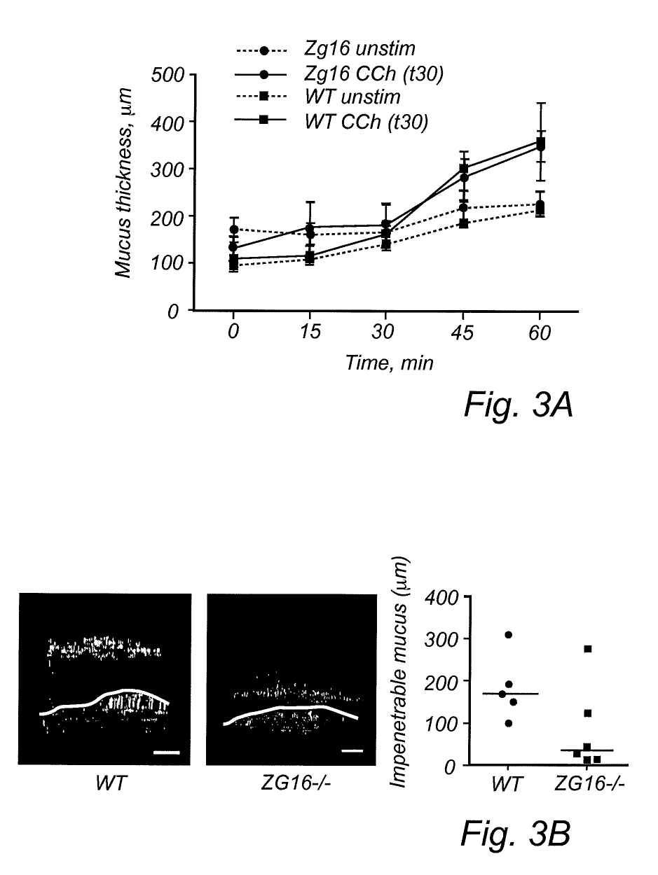

First the mucus layer was measured by mounting distal colon tissue in a horizontal perfusion chamber. The mucus growth was monitored over a period of 60 min with or without the addition of the secretagogue carbachol and compared to WT controls (FIG. 3A). The measurements showed no difference between the two groups. To access the quality of the mucus layer, bacterial sized beads were applied to the mucus layer of explants and the penetrability of the beads monitored by confocal microscopy (FIG. 3B). The WT mice all showed a spatial separation of the beads from the epithelia of at least 100 μm. In the Zg16^{-/-} mice more beads were found closer to the epithelium showing a tendency of a more penetrable mucus, the difference was however not significant different using a Mann-Whitney U-test. To further examine the effect of lacking Zg16, Carnoy fixed distal colon sections with fecal material were examined (not shown). Hematoxylin and eosin stained tissue sections showed normal histology with no signs of inflammation such as crypt elongation and neutrophil infiltration. Alcian blue and Periodic Acid-Schiff stains carbohydrates therefore useful to stain the heavily glycosolated mucins, but no difference could be detected. The sections were also stained with the bacterial stain Brown-Brenn since to evaluate if the absence of Zg16 altered the bacterial localization. Brown-Brenn stains Gram positive bacteria purple and Gram negative pink. The sections show bacteria close to the epithelium in the Zg16^{-/-} animals compared to the WT were the bacteria is separated by mucus also staining pink. Sections was stained with an anti-Muc2 antiserum as well as with an antibody that reacts with lipoteichoic acid (LTA) present on Gram positive bacteria. The inner mucus layer in the Zg16^{-/-} was not as well organized as the WT inner mucus. There were also more bacteria in the inner mucus layer revealed both by the DNA and the LTA stains.

Combined, the results indicate that Zg16^{-/-} mice has a normal mucus thickness, but a higher bacterial penetrability allowing the bacteria to come closer to the epithelium.

### Example 4

### ZG16 Alters Distal Colonic Mucus Penetration by Gram-Positive Bacteria Ex-Vivo

To explore how ZG16 binding might affect bacterial interactions with the mucus layer, ex-vivo confocal imaging was used to analyse the distribution of rZG16 treated bacteria in WT distal colonic mucus.

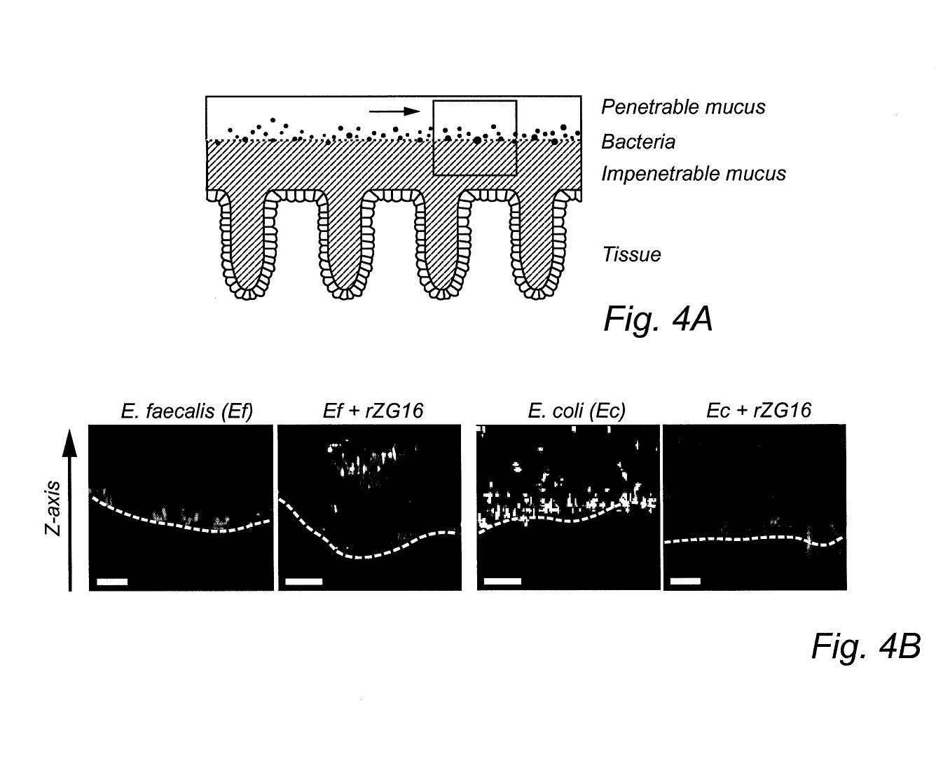

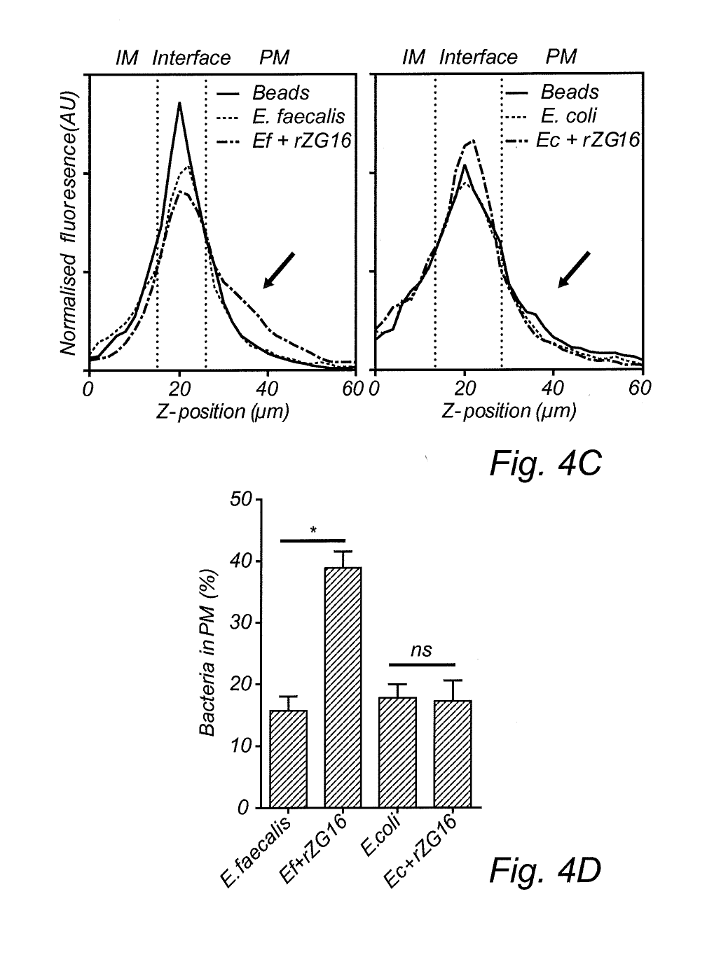

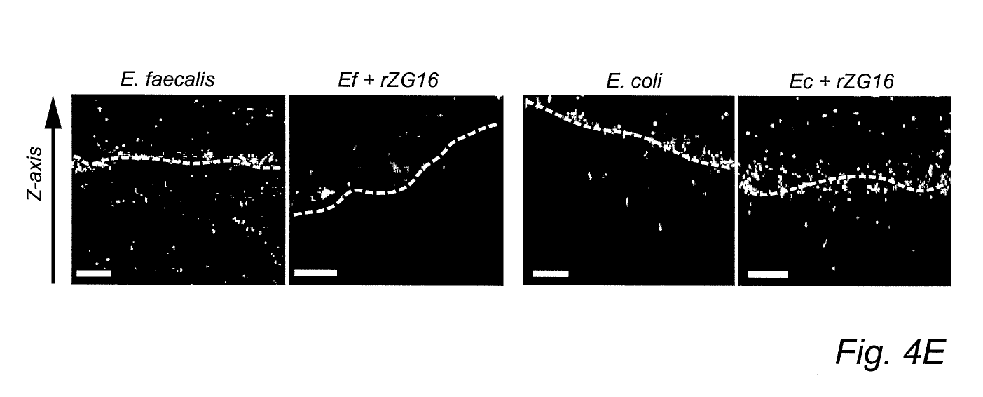

Distal colon tissue was mounted in a confocal imaging chamber and 2 μm fluorescent beads were apically applied in order to mark the interface between the impenetrable (IM) and penetrable (PM) mucus layers. G-positive commensal bacterium Enterococcus faecalis and Gram-negative Eschercia. coli were fluorescently labelled, treated with rZG16 and applied to the colonic mucus. Confocal z-stacks were acquired over the IM/PM interface as illustrated in FIG. 4A. Under control conditions the majority of bacterial cells settled at the IM/PM interface of the WT mucus; however, rZG16 exposure resulted in aggregated bacterial cells which did not fully penetrate the PM layer (FIG. 4B). This effect was reproducible and resulted in a shift in bacterial distribution with a significantly higher proportion of rZG16 treated G+bacteria, but not G−, present in the PM layer when compared to untreated controls (FIGS. 4C and 4D). This shows that the previously described ZG16-mediated aggregation effect can alter the capacity of bacteria to penetrate the colonic mucus. To further explore the role of ZG16 the same method was applied using tissue from Zg16^{-/-} mice. The IM layer of the Zg16^{-/-} colonic mucus was more penetrable to both beads (as previously noted) and bacteria under control conditions, but rZG16 treated bacteria were mostly unable to penetrate the IM layer (FIG. 4E). Interestingly the inhibition of bacterial IM layer penetration by rZG16 was not limited to aggregated cells as individual cells also failed to penetrate the IM layer despite its remaining penetrability to beads of a similar size.

This suggests that ZG16 can exclude bacteria from the IM layer by aggregation-independent mechanisms.

### Example 5

### ZG16 Alters the Distribution, Composition, and Mobility of the Mucus Associated Intestinal Microbiota

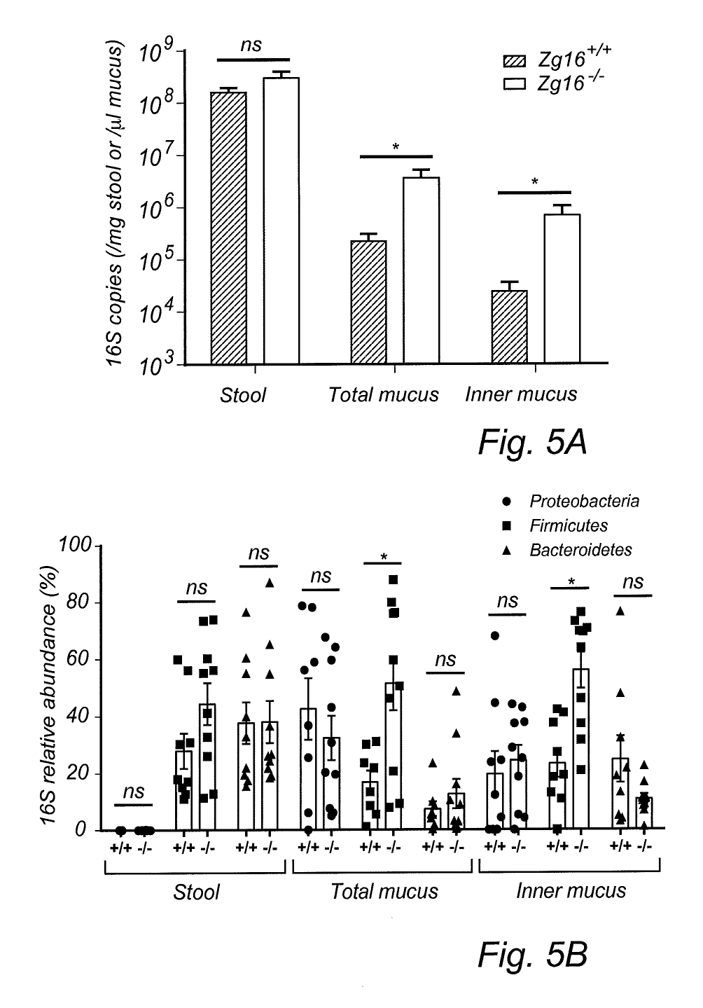

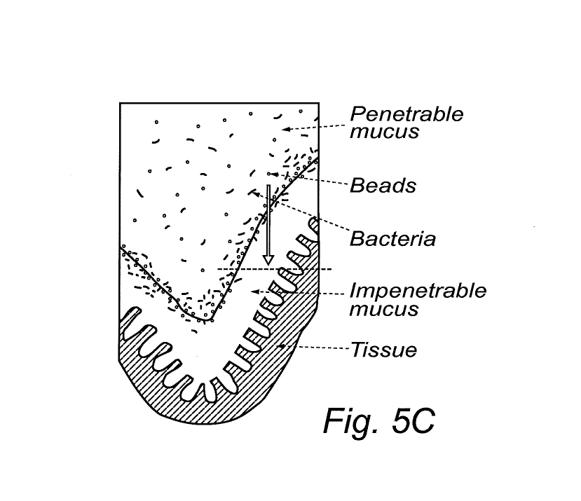

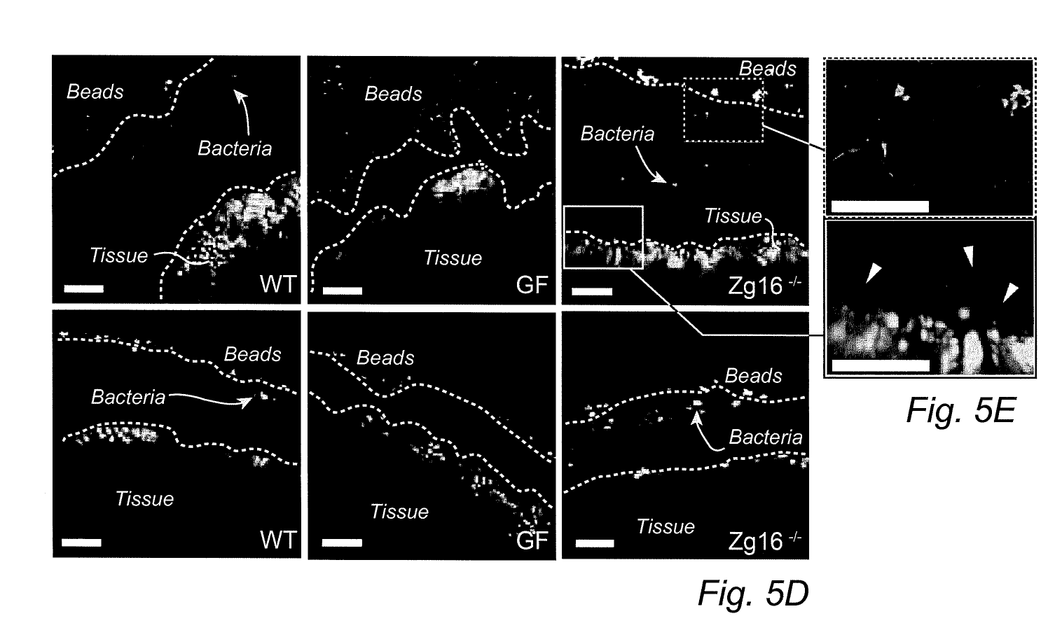

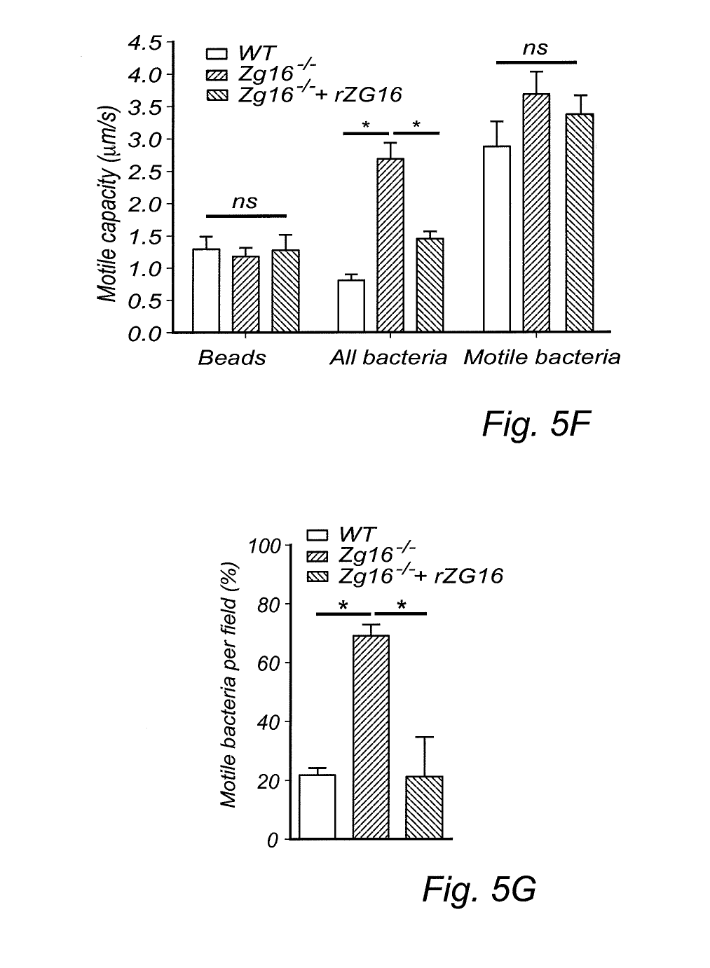

DNA was extracted from stool and biopsy punch collected tissues from unflushed (total mucus) or flushed (inner mucus) distal colon from littermate Zg16^{+/+} and Zg16^{+/-} mice and bacterial 16S was quantified by qPCR and normalized to stool mass or mucus thickness. The amount of bacteria/μl mucus was significantly higher in the Zg16^{-/-} total and inner mucus compared to Zg16^{+/+} (FIG. 5A). However, no differences were observed in the stool. Analysis of the relative abundance of 16S from the phyla Proteobacteria, Firmicutes and Bacteroidetes by qualitative qPCR showed significant increases in the Gram-positive Firmicutes, but neither of the Gram-negative phyla, in the mucus samples. Again, no differences were observed in stool samples (FIG. 5B).

To investigate the effect of ZG16 on the distribution of the microbiota within the mucus in vivo, unflushed distal colon tissue was mounted in an imaging chamber with green fluorescent beads to mark the IM/PM interface (FIG. 5C). The Syto9 nucleic acid-binding dye (red) was used to stain the host tissue and microbiota in situ. Confocal microscopy revealed a band of Syto9 stained objects in the mucus of WT (conventionally raised) mice that resembled morphologically diverse bacterial cells, and these objects were absent in GF (germ-free) mucus (FIG. 5D, E). In WT mucus the microbiota was typically found in a discrete zone close to the IM/PM interface with limited bacteria deeper within the IM (FIG. 5D). The Zg16^{-/-} IM mucus contained notably more bacterial cells than the WT mucus with bacterial cells observed at the tissue surface (FIG. 5D, E).

As it was noted that mucus-associated bacteria in the Zg16^{-/-} mucus appeared more motile, the motile capacity (MC; the maximum speed observed for an individual bacterium) of all detected bacteria was analyzed by live imaging of bacteria in the mucus. The WT microbiota was mostly static, with a mean MC equal to the background movement of beads in the mucus, whereas the mean MC of the Zg16^{-/-} microbiota was significantly higher (FIG. 5F). Analysis of only motile bacteria in WT and Zg16^{-/-} mucus demonstrated that the MC difference was due to an increase in the proportion of motile bacteria in the Zg16^{-/-} mucus rather than overall bacterial velocity (FIG. 5F,G). Incubation of Zg16^{-/-} colonic mucus with rZG16 restored the mean bacterial MC and proportion of motile bacteria to WT levels (FIG. 5F, G). Together this suggests that ZG16 works together with the mucin network and forms an enhanced barrier to Gram-positive colonization of the IM layer. Trapping of bacteria and reduced motility in the mucus may be the mechanistic explanation of ZG16's contribution to colonic mucus barrier.

### Example 6

### ZG16^{-/-} Mice Showed Increased Host Systemic Bacterial Load, Serum Cytokine Levels, Body Fat and Body Mass

In light of evidence indicating a barrier function-related role for ZG16 in the colon it seemed plausible that animals lacking this protein would be at higher risk of increased bacterial translocation across the intestinal barrier and subsequent microbial dissemination to systemic tissues.

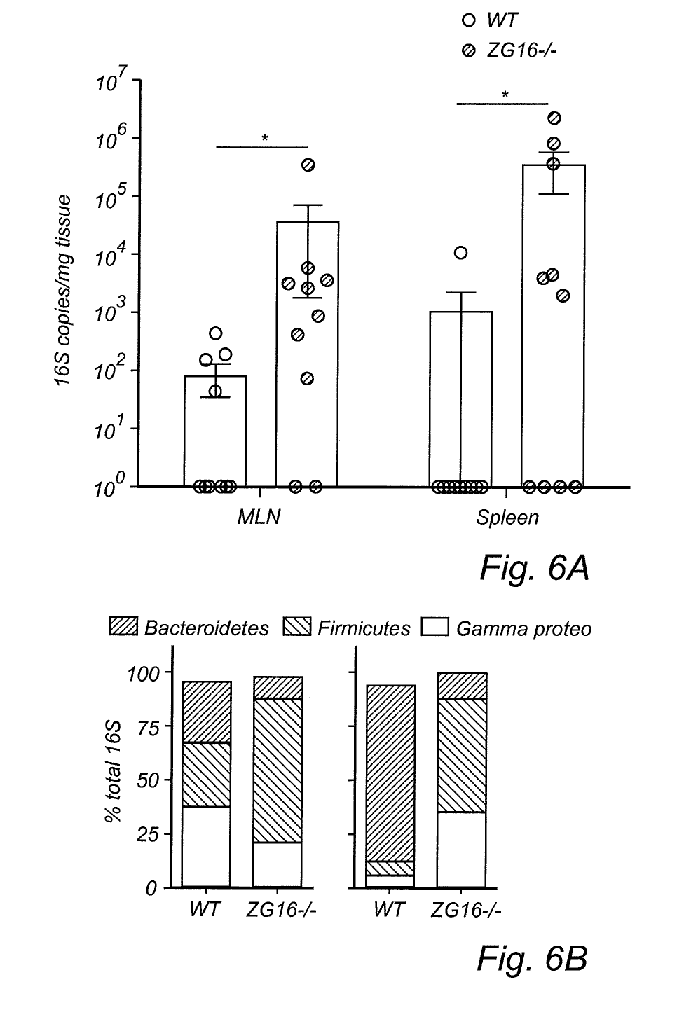

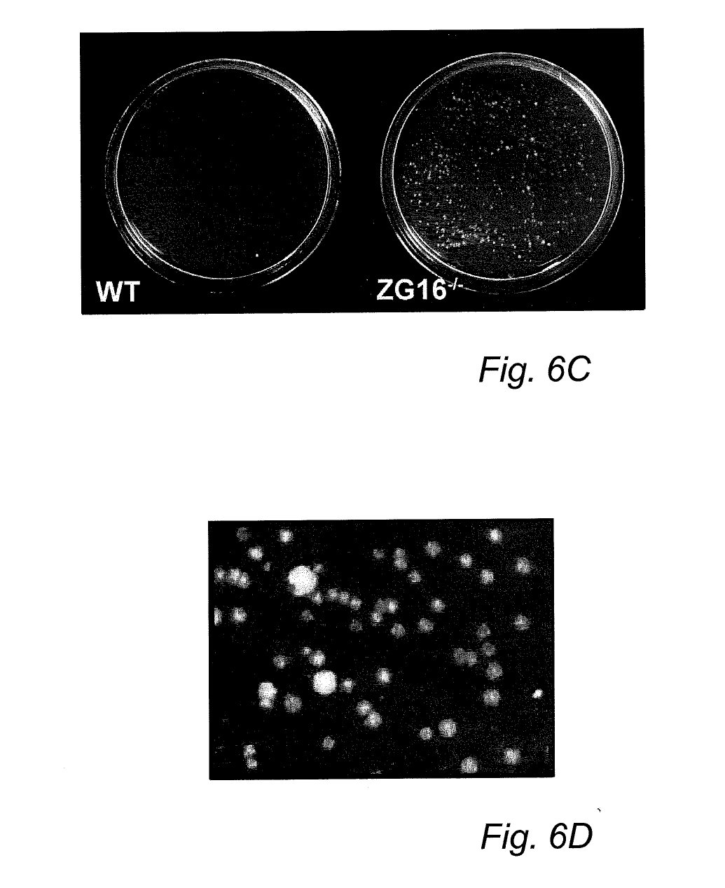

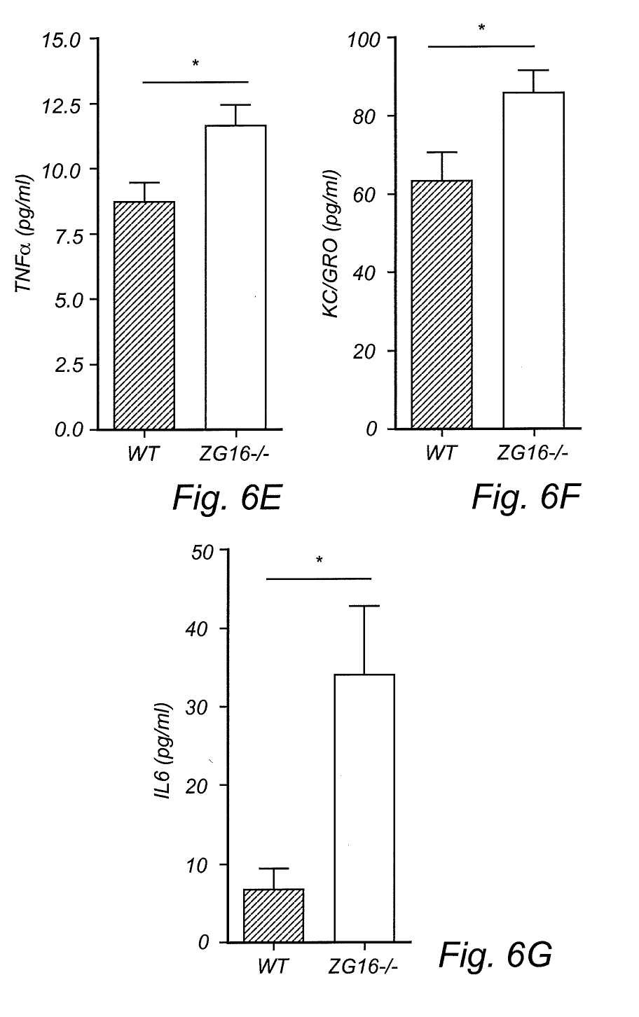

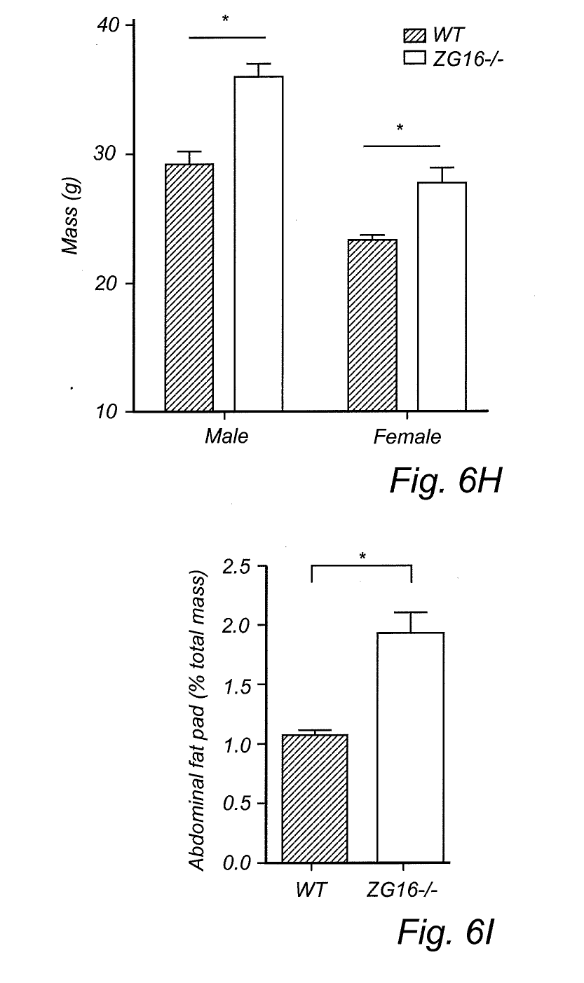

To assess this risk, systemic tissues that might be exposed to bacteria which escape intestinal confinement were sampled from WT and Zg16^{-/-} mice and their bacterial load quantified by 16S qPCR of extracted DNA. The 16S load was significantly higher in mesenteric lymph node (MLN) and spleen tissues recovered from ZG16^{-/-} compared to WT mice (FIG. 6A). Qualitative analysis of 16S DNA amplified from all Zg16^{-/-} systemic tissues showed that the dominant taxonomic group was the Gram-positive phylum Firmicutes (FIG. 6B). In contrast, 16S detected in WT tissues had a more variable taxonomic profile. In order to assess the viability of systemic bacteria detected by qPCR, tissue sample homogenates were cultured to check for viable bacterial growth. Viable bacteria were cultured from all Zg16^{-/-} systemic tissues including the spleen which, of the three tissue types sampled, is the most anatomically distant from the intestine (FIG. 6C). Furthermore, multiple colony morphotypes were observed in bacterial cultures thus demonstrating the polymicrobial nature of the bacteria present in these tissues (FIG. 6D). These results strongly indicated that, in contrast to the WT animals, multiple bacterial species had penetrated the systemic tissues in most of the Zg16^{-/-} mice. In immunocompetent animals the systemic presence of viable bacteria should be accompanied by indications of immune system activity. We therefore prepared serum from WT and Zg16^{-/-} whole blood in order to compare levels of a range of immunomodulatory cytokines. Serum titres of three pro-inflammatory cytokines, IL-6, KC/GRO (murine IL-8 homologue) and TNFα were marginally, but significantly elevated in Zg16^{-/-} serum compared to WT controls (FIG. 6E-G). No significant changes were observed in any of the other cytokines examined. Finally, increased intestinal permeability and low grade inflammation are known to correlate with increased body and fat mass. Body mass comparison of age matched WT and Zg16^{-/-} mice demonstrated a 16.7% and 20.2% increased mass in male and female Zg16^{-/-} mice, respectively (FIG. 6H). The Zg16^{-/-} also showed an increased fat mass (FIG. 6I).

Consequently, mice lacking ZG16 display several features associated with intestinal barrier dysfunction including systemic inflammation, obesity and increased fat deposits. This further support a crucial role for ZG16 in maintaining a colon bacterial barrier.

### Example 7

### Trypsin Resistance of ZG16

The resistance of ZG16 to digestive enzymes was investigated in order to establish whether it would be possible to administer ZG16 via an oral route. The digestive enzyme trypsin did not digest ZG16 into small peptides except for the release of a recombinant addition by the non-ZG16 part VYPTSCSRCSRGSDDDDK. Peptides from both the N-terminal and C-terminal was detected in the treated sample showing that ZG16 is trypsin resistant.

### Summary of Results and Conclusions of Examples 1-7

ZG16 binds relatively strongly to bacterial peptidoglycan.
ZG16 binds to intact G+ bacteria such as Lactobacillus jensenii.
ZG16 binds to bacteria, but do not have any antimicrobial activity by itself.
ZG16 does not affect the viability of bacteria after binding, instead ZG16 cause aggregation.
Zg16−/− mice have a normal mucus thickness, but a higher bacterial penetrability allowing the bacteria to come closer to the epithelium.
ZG16 can exclude bacteria from the impenetrable mucus layer by aggregation-independent mechanisms.
ZG16 together with the mucin network forms an improved barrier to Gram-positive colonisation of the IM layer. Bacterial trapping and reduced motility in the mucus may be the mechanistic explanation.
Mice lacking ZG16 display several features associated with intestinal barrier dysfunction.
The more penetrable mucus allows more bacteria to translocate to systemic tissues, something that may trigger e.g. a systemic inflammation and obesity.
ZG16 is resistant to trypsin degradation, allowing for administration via an oral route.

Altogether, the results of the extensive experimentation underlying the present invention provide far-reaching evidence for the crucial role of ZG16 in maintaining a colon barrier, and strongly substantiate the finding that administration of ZG16 will lead to aggregation of bacteria and inhibition of mucus penetration. This in turn substantiates the pharmaceutical effects provided by ZG16 in the treatment of disorders related to a bacterial imbalance in the gastrointestinal tract, in particular disorders caused by Gram-positive bacteria coming into contact with the gastrointestinal epithelium.

1.-10. (canceled)

11. A method for prevention and/or treatment of a disorder related to a bacterial imbalance in the gastrointestinal tract selected from a gastrointestinal disease, a bacterial infection, and/or a disorder related to the metabolic syndrome, comprising administering to a patient in need thereof a pharmaceutically effective amount of a protein based on ZG16 comprising a sequence motif defined by any one of the sequences having SEQ ID NO:s 1-3, optionally excluding the amino acids in positions 1-20, or 1-16.

12. A method for prevention and/or treatment of a disorder related to a bacterial imbalance in the gastrointestinal tract selected from a gastrointestinal disease, a bacterial infection, and/or a disorder related to the metabolic syndrome comprising administering to a patient in need thereof a pharmaceutically effective, amount of a protein based on ZG16 having a sequence identity of at least 80% with human ZG16 as defined by SEQ ID NO. 6, optionally excluding the amino acids in positions 1-20, or 1-16.

13. A method for prevention and/or treatment of a disorder related to a bacterial imbalance in the gastrointestinal tract selected from a gastrointestinal disease, a bacterial infection, and/or a disorder related to the metabolic syndrome, comprising administering to patient in need thereof a pharmaceutically effective amount of a protein based on ZG16 defined by SEQ ID NO:s 5-8, optionally excluding the amino acids in positions 1-20, or 1-16.

14. he method according to claim wherein the gastrointestinal disease is IrritableBowel Syndrome (IBS), or a gasitrointestinal inflammatory disease.

15. The method according to claim 14, wherein the gastrointestinal inflammatory disease is a systemic inflammatory disease, or Inflammatory Bowel Disease (IBD).

16. The method according to claim 11, wherein said gastrointestinal disease is intestinal symptoms associated with cystic fibrosis.

17. The method according to claim 11, wherein said disorder related to the metabolic syndrome is obesity and/or diabetes.

18. A pharmaceutical composition comprising a pharmaceutically effective amount of a protein based on ZG16 comprising a sequence motif defined by any one of the sequences having SEQ ID NO:s 1-3, optionally excluding the amino acids in positions 1-20, or 1-16.

19. A pharmaceutical composition comprising a pharmaceutically effective amount of a protein based on ZG16 having a sequence identity of at least 80% with human ZG16 as defined by SEQ ID NO. 6, optionally excluding the amino acids in positions 1-20, or 1-16.

20. A pharmaceutical composition comprising a pharmaceutically effective amount of a protein based on ZG16 defined by SEQ ID NO:s 5-8, optionally excluding the amino acids in positions 1-20, or 1-16.

21. The pharmaceutical composition according to claim 18, wherein said pharmaceutical composition is formulated for oral, nasal, rectal, topical, intravenous, intraartery, intracavitary, intramuscular, subcutaneous, or transdermal administration.

22. The pharmaceutical composition according to claim 18, wherein said pharmaceutical composition is in the form of a tablet, a capsule a suppository, a powder, a paste, a solution, a cream, a gel, and/or an administration unit comprising bacteria having the ability to produce and secrete the protein based on ZG16.

23. A method for the prevention and/or treatment of a disorder which is related to a bacterial imbalance in the gastrointestinal tract, comprising administering to a patient in need thereof a pharmaceutically effective amount of a protein based on ZG16.

24. A method according to claim 23, wherein the protein based on ZG16 comprises a sequence motif defined by any one of the sequences having SEQ ID NO:s 1-3, optionally excluding the amino acids in positions 1-20, or 1-16.

25. A method according to claim 23, wherein protein based on ZG16 has a sequence identity of at least 80% with human ZG16 as defiir ed by SEQ ID NO, 6, optionally excluding the amino acids in positions 1-20, or 1-16.

26. A method according to claim 23, wherein the protein based on ZG16 is defined by SEQ ID NO:s 5-8, optionally excluding the amino acids in positions 1-20, or 1-16.

27. A method according to claim 23, wherein the disorder is selected from a gastrointestinal disease, a bacterial infection, and/or a disorder related to the metabolic syndrome.
